(19) United States
(12) Patent Application Publication
(10) Pub. No.: US 2020/0392455 A1
(43) Pub. Date: Dec. 17, 2020

(54) NERVE CELL PRODUCTION METHOD

(71) Applicants: I Peace, Inc., Palo Alto, CA (US); ID Pharma Co., Ltd., Tokyo (JP)

(72) Inventors: Koji TANABE, Palo Alto, CA (US); Makoto INOUE, Tokyo (JP); Tsugumine SHU, Tokyo (JP); Toyotaka MORI, Tokyo (JP)

(73) Assignees: I Peace, Inc., Palo Alto, CA (US); ID Pharma Co., Ltd., Tokyo (JP)

(21) Appl. No.: 16/768,526

(22) PCT Filed: Nov. 27, 2018
(86) PCT No.: PCT/JP2018/043573

§ 371 Date: May 29, 2020

Related U.S. Application Data
(60) Provisional application No. 62/592,629, filed on Nov. 30, 2017.

(51) Int. Cl. C12N 5/0793

(52) U.S. Cl. CPC C12N 5/0619; C12N 2501/13; C12N 2506/02; C12N 2510/02; C12N 2506/45; C12N 2501/60

(57) ABSTRACT

Provided is a nerve cell production method including preparing stem cells, and introducing Sendai virus by infection to the stem cells to induce the stem cells into nerve cells by allowing the Sendai virus to express mRNA which synthesizes an inducing factor in the stem cells.

### FIELD

The present invention relates to cell technology, and to a nerve cell production method.

### BACKGROUND

Induced pluripotent stem cells (iPS cells) are capable of being transformed into numerous types of cells composing the body. Therefore, iPS cells capable of being transformed into various types of somatic cells or tissues are considered promising for use in cell graft therapy and innovative drug development and research. In 2014, for example, retina cells produced from iPS cells were successfully applied in transplantation therapy. Projects are being pursued not only in Japan but throughout the world for creating brain cells and different organ cells from iPS cells for use in transplantation therapy.

Numerous methods for transforming iPS cells to differentiated cells exist in the prior art. For utilization of iPS cells in transplantation therapy, however, it is important to establish highly efficient inducing methods for iPS cells. Specifically, it is necessary to establish techniques to be used for inducing differentiation of iPS cells to differentiated cells, improving the efficiency and precision of induction and ensuring that the functionality of the created differentiated cells is able to withstand transplantation therapy.

Methods for inducing differentiated cells from iPS cells or embryonic stem cells (ES cells) to somatic cells have conventionally included methods that imitate the process of development, by combining hormones or growth factors that are the determinants of the properties of the cells, as well as low molecular compounds, and varying their quantity ratios or concentrations with time. However, it is difficult to completely emulate the development process in vitro, and efficiency is also poor. Moreover, induction of human somatic cells requires a much longer inducing period than for mice, with conventionally 3 months or longer, for example, being necessary to prepare mature nerves.

Another problem is that inducing efficiency differs widely depending on the line of ES/iPS cells, while the properties of induced somatic cells are non-homogeneous. When chemical substances have actually been added to different multiple ES cell clones to create various types of cells, it has been demonstrated that certain clones exist that are readily induced to pancreas cells or that are readily induced to heart cells, and therefore that different clones have varying induction potencies (see NPL 1, for example). In addition, it has been demonstrated that when the method known as a serum-free suspension culture method (SFEBq method) is used, in which iPS cells are cultured in medium free of serum or of chemical substances that inhibit neuron induction, to produce neurons from iPS cells/ES cells, thereby producing neurons from dozens of lines of iPS cells, some of the iPS/ES cell clones present are difficult to transform into neurons (see NPL 2, for example).

Specifically, cells which are induced from human ES/iPS cells by methods utilizing hormones or chemical substances have been confirmed to be fetal-stage somatic cells in the initial stages. It is extremely difficult to induce to mature human somatic cells, and their culturing requires long periods of several months. However, for drug development and transplant medicine for fully developed individuals, it is important to prepare somatic cells that match the maturation level of the individual.

For neurons, which include cells of a variety of different subtypes, it is not possible to induce to neuronal subtypes in a uniform manner from ES/iPS cells by methods utilizing hormones or chemical substances. Therefore, drug screening specific for designated neuronal subtypes is not possible. This lowers the efficiency for drug screening. For transplant medicine as well, it is not possible to concentrate and transplant only specific diseased cells.

For this reason, methods have been proposed wherein genes for the properties of specific somatic cells are directly transferred into ES/iPS cells using deoxyribonucleic acid (DNA) viruses which are integrated into the genome, to create the desired somatic cells. Methods using deoxyribonucleic acid (DNA) viruses which are integrated into the genome allow specific generation of mature neurons in very short time periods compared to methods using hormones or chemical substances, such as 2 weeks, for example. Moreover, generating neurons by specific gene transfer allows excitatory nerves alone, for example, to be obtained in a homogeneous manner Therefore, specific drug screening for specific neuronal subtypes becomes possible, potentially making it possible to concentrate and transplant only cells specific to a disease, for transplant medicine.

However, in methods of inducing differentiation of stem cells to somatic cells using deoxyribonucleic acid (DNA) viruses which are integrated into the genome to cause expression of specific genes, the genes are inserted in the genome of the ES/iPS cells, causing damage to the endogenous genes. This has resulted in problems such as failure to properly accomplish drug screening, and the risk of canceration of grafts (see NPLs 3 and 4, for example).

### CITATION LIST

### Non-Patent Literature

NPL 1: Nature Biotechnol. 26(3): 313-315, 2008.

NPL 2: PNAS, 111:12426-12431, 2014

NPL 3: N Eng J Med, 346:1185-1193, 2002

NPL 4: Science 302: 415-419, 2003

### SUMMARY

### Technical Problem

It is an object of the present invention to provide a nerve cell production method that allows nerve cells to be produced efficiently in a short period of time, without damaging cellular genes.

### Solution to Problem

According to one aspect of the present invention there is provided a nerve cell production method that includes preparing stem cells, and introducing Sendai virus by infection to the stem cells to induce the stem cells into nerve cells by allowing the Sendai virus to express messenger ribonucleic acid (mRNA) that synthesizes inducing factors in the stem cells.

In the nerve cell production method, the stem cells may be artificial pluripotent stem cells (iPS cells).

In the nerve cell production method, the stem cells may be embryonic stem cells (ES cells).

In the nerve cell production method, the inducing factors may include neurogenin (NGN). The inducing factors may include only neurogenin (NGN).

In the nerve cell production method, the inducing factors may include achaete-scute homolog (ASCL).

In the nerve cell production method, the inducing factors may include myelin transcription factor (MYT).

In the nerve cell production method, the inducing factors may include distal-less homeobox (DLX).

In the nerve cell production method, the nerve cells may be neurons, neural stem cells, or neural precursor cells. The neurons may be of one or more types selected from the group consisting of excitatory neurons, inhibitory neurons, dopamine-producing neurons, locomotor neurons, and cerebral neurons. The nerve cells may be oligodendrocyte precursor cells and oligodendrocytes.

In the nerve cell production method, the nerve cells may be positive for one or more of NGN gene, ASCL gene, MYT gene, and DLX gene.

In the nerve cell production method, the nerve cells may be positive for one or more selected from the group consisting of tyrosine hydroxylase (TH), TUJ1, BRN2, NGN, Tubulin, MAP2, PSA-NCAM, vGLUT, SATB2, chAT, HB9, MUNC13, HOMER1, vGAT, and GAD65/67.

In the nerve cell production method, the Sendai virus may be allowed to express mRNA of a drug resistance gene in the stem cells.

In the nerve cell production method, the drug resistance gene may be one or more selected from the group consisting of puromycin resistance gene, blasticidin resistance gene, hygromycin resistance gene, and neomycin resistance gene.

The nerve cell production method may include selecting cells exhibiting drug resistance after infection of the stem cells with Sendai virus.

The nerve cell production method may include selecting cells exhibiting drug resistance of one or more selected from the group consisting of puromycin resistance, blasticidin resistance, hygromycin resistance, and neomycin resistance, after infection of the stem cells with Sendai virus.

In the nerve cell production method, the density of the stem cells at the infection with Sendai virus may be from 0.2×10⁵ cells/well to 1.0×10⁶ cells/well in the wells of a 12-well plate.

In the nerve cell production method, the multiplicity of infection (MOI) of Sendai virus may be from 0.1 to 100.0 per time.

In the nerve cell production method, the culture medium when the stem cells are infected with Sendai virus may be one selected from mTeSR^{R}1, TeSR2^{R}, and Stem Fit.

### Advantageous Effects of Invention

According to the invention, it is possible to provide a nerve cell production method that allows nerve cells to be produced efficiently in a short period of time, without damaging cellular genes.

### BRIEF DESCRIPTION OF DRAWINGS

FIG. 1 is a microscope photograph of the cells in Example 1.

FIG. 2 is a fluorescence microscope photograph of the cells fluorescent-labeled using anti BRN2 antibodies in Example 1.

FIG. 3 is a fluorescence microscope photograph of the cells fluorescent-labeled using anti HOMER1 antibodies in Example 2.

FIG. 4 is a fluorescence microscope photograph of the cells fluorescent-labeled using anti MAP2 antibodies in Example 3.

FIG. 5 is a fluorescence microscope photograph of the cells fluorescent-labeled using anti MUNC13-1 antibodies in Example 4.

FIG. 6 is a fluorescence microscope photograph of the cells fluorescent-labeled using anti SATB2 antibodies in Example 5.

FIG. 7 is a fluorescence microscope photograph of the cells fluorescent-labeled using anti TUJ1 antibodies in Example 6.

FIG. 8 is a fluorescence microscope photograph of the cells fluorescent-labeled using anti vGLUT antibodies in Example 7.

FIG. 9 is a fluorescence microscope photograph of the cells fluorescent-labeled using anti chAT antibodies in Example 8.

FIG. 10 is a fluorescence microscope photograph of the cells fluorescent-labeled using anti HB9 antibodies in Example 9.

FIG. 11 is a fluorescence microscope photograph of the cells fluorescent-labeled using anti TH antibodies in Example 10.

FIGS. 12(a) and 12(b) are microscope photographs of the cells in Example 11. FIG. 12(c) is a fluorescence microscope photograph of the cells fluorescent-labeled using anti TUJ1 antibodies in Example 11.

FIG. 13 is a fluorescence microscope photograph of the cells fluorescent-labeled using anti BRN2 antibodies in Example 12.

FIG. 14 is a fluorescence microscope photograph of the cells fluorescent-labeled using anti GAD65/67 antibodies in Example 13.

FIG. 15 is a fluorescence microscope photograph of the cells fluorescent-labeled using anti MAP2 antibodies in Example 14.

FIG. 16 is a fluorescence microscope photograph of the cells fluorescent-labeled using anti vGAT antibodies in Example 15.

FIG. 17 is a fluorescence microscope photograph of the cells fluorescent-labeled using anti TUJ1 antibodies in Example 16.

FIG. 18 is a fluorescence microscope photograph of the cells fluorescent-labeled using anti MAP2 antibodies in Example 17.

FIG. 19 is a fluorescence microscope photograph of the cells fluorescent-labeled using anti BRN2 antibodies in Example 18.

FIG. 20 is a fluorescence microscope photograph of the cells fluorescent-labeled using anti vGAT antibodies in Example 19.

FIG. 21 is a fluorescence microscope photograph of the cells fluorescent-labeled using anti GAD65/67 antibodies in Example 20.

FIG. 22 is a fluorescence microscope photograph of the cells fluorescent-labeled using anti TH antibodies in Example 21.

FIG. 23 is a fluorescence microscope photograph of the cells fluorescent-labeled using anti chAT antibodies in Example 22.

FIG. 24 is a fluorescence microscope photograph of the cells fluorescent-labeled using anti vGLUT antibodies in Example 23.

FIG. 25 is a fluorescence microscope photograph of the cells fluorescent-labeled using anti SATB2 antibodies in Example 24.

FIG. 26 is a graph showing the results of flow cytometry in Example 25.

### DESCRIPTION OF EMBODIMENTS

Embodiments of the invention will now be explained in detail. The embodiments described below are merely examples of devices and methods for implementing the technical concept of the invention, and the technical concept of the invention is not limited to the described combinations of structural components. The technical concept of the invention may incorporate various modifications such as are within the scope of the Claims.

The method of producing somatic cells from stem cells according to the embodiments of the invention includes preparing stem cells, and introducing Sendai virus by infection to the stem cells to induce the stem cells into nerve cells by allowing the Sendai virus to express mRNA that synthesizes inducing factors in the stem cells.

Both induced pluripotent stem cells (iPS cells) and embryonic stem cells (ES cells) can be used as stem cells. The stem cells may be human stem cells or non-human animal stem cells.

Induction refers to reprogramming, initialization, transformation, differentiation transformation (Transdifferentiation or Lineage reprogramming), differentiation induction, cell f ate reprogramming, and the like.

Examples of nerve cells to be induced include neurons, neural stem cells and neural precursor cells. Examples of neurons include excitatory neurons, inhibitory neurons, dopamine-producing neurons, locomotor neurons, and cerebral neurons. Alternatively, the nerve cells may be oligodendrocyte precursor cells, oligodendrocytes, and the like.

The Sendai virus (SeV) introduced into the stem cells has an RNA genome which expresses mRNAs to synthesize inducing factors which induce stem cells into neurons. Incidentally, production of recombinant Sendai virus vectors capable of expressing any arbitrary mRNA may be commissioned to, for example, ID Pharma Co., Ltd. using patented technology. mRNAs for synthesizing inducing factors include, for example, mRNAs of NGN genes such as NGN2, mRNAs of ASCL genes such as ASCL1, mRNAs of MYT genes such as MYT1L, and mRNAs of DLX genes such as DLX2. NGN2, ASCL1, MYT1L and DLX2 are switching proteins required for generating neurons. NGN2 is capable of generating excitatory neurons. Each of ASCL1, MYT1L, or DLX2 is capable of generating inhibitory neurons. The combination of ASCL1 and MYT1L is capable of generating excitatory neurons, inhibitory neurons, and dopamine-producing neurons.

For example, the Sendai viruses introduced into the stem cells express one or more selected from mRNAs of NGN genes such as NGN2, mRNAs of ASCL genes such as ASCL1, mRNAs of MYT genes such as MYT1L, and mRNAs of DLX genes such as DLX2, as mRNAs synthesizing inducing factors.

The Sendai viruses may have an RNA genome expressing mRNAs of drug resistance genes. Examples of drugs include antibiotics such as puromycin, blasticidin, hygromycin, neomycin, G418, and zeocin. For example, the Sendai viruses introduced into the stem cells express mRNAs of drug resistance genes. The cells in which mRNAs of drug resistance genes are expressed exhibit drug resistance.

Cells which exhibit drug resistance after infection may be selected when the Sendai viruses are allowed to express mRNAs of drug resistance genes. For example, when one or more of the mRNAs of puromycin-resistant genes, blasticidin-resistant genes, hygromycin-resistant genes, neomycin-resistant genes, G418-resistant genes, and zeocin^{R}-resistant genes are expressed by Sendai viruses, cells in which the Sendai viruses are introduced can be screened by exposing the cells after infection to the corresponding antibiotic and killing the cells other than the ones into which the Sendai viruses have been introduced.

The Sendai viruses may have, for example, RNAs capable of expressing mRNAs including mRNAs of NGN2 gene and mRNAs of puromycin resistance gene (hereinafter referred to as “NGN2-Puro mRNA(s)”). The sequence of the mRNAs of NGN2 gene corresponds, for example, to the DNA sequence shown as SEQ ID NO.1 (murine) or SEQ ID NO.2 (human) The cells in which NGN2-Puro mRNAs are expressed produce NGN2 and exhibit puromycin resistance.

The Sendai viruses may have, for example, RNAs capable of expressing mRNAs including mRNAs of ASCL1 gene, mRNAs of MYT1L gene, and mRNAs of puromycin resistance gene (hereinafter referred to as “ASCL1-MYT1L-Puro mRNA(s)”). The sequence of the mRNAs of ASCL1 gene corresponds, for example, to the DNA sequence shown as SEQ ID NO.3 (murine) or SEQ ID NO.4 (human) The sequence of the mRNAs of MUT1L gene corresponds, for example, to the DNA sequence shown as SEQ ID NO.5 (murine), SEQ ID NO.6 (murine), SEQ ID NO.7 (murine), or SEQ ID NO.8 (human) The cells in which ASCL1-MYT1L-Puro mRNAs are expressed produce ASCL1 and MYT1L and exhibit puromycin resistance.

The Sendai viruses may have, for example, RNAs capable of expressing mRNAs including mRNAs of DLX2 gene and mRNAs of blasticidin resistance gene (hereinafter referred to as “DLX2-Blast mRNA(s)”). The sequence of the mRNAs of DLX2 gene corresponds, for example, to the DNA sequence shown as SEQ ID NO.9 (murine) or SEQ ID NO.10 (human). The cells in which DLX2-Blast mRNAs are expressed produce DLX2 and exhibit blasticidin resistance.

The Sendai viruses are introduced into the stem cells by being suspended in the cell culture medium. The Sendai viruses recognize the cell surface antigens and infects the stem cells.

The density of the stem cells at the infection with Sendai virus is, for example, from 0.2×10⁵ cells/well to 1.0×10⁶ cells/well, from 0.5×10⁵ cells/well to 8.0×10⁵ cells/well, or from 1.0×10⁵ cells/well to 4×10⁵ cells/well, in the wells of a 12-well plate.

The titer of the Sendai viruses used is, for example, from 1×10¹² CIU/mL to 1×10⁵ CIU/mL, from 1×10¹⁰ CIU/mL to 1×10⁶ CIU/mL, or from 1×10⁹ CIU/mL to 1×10⁷ CIU/mL. The multiplicity of infection (MOI) of the Sendai viruses is, for example, from 0.1 to 100.0, from 1.0 to 50.0, or from 1.0 to 20.0 per time.

The culture medium used at the infection with Sendai virus is, for example, stem cell culture medium such as mTeSR1^{R}, TeSR2^{R} (STEMCELL Technologies Inc.), and Stem Fit (ReproCELL Inc.). After infection with Sendai virus, the culture medium may be replaced with a culture medium suitable for induction to nerve cells (hereinafter also referred to as “neural induction culture medium”). Examples for a neural induction culture medium include NDiff 227^{R} (TAKARA BIO INC.), Neurobasal^{R} (Thermo Fisher Scientific Inc.), Human ES/iPS Neuronal Differentiation Medium (Merck KGaA), and N3 culture medium. N3 culture medium is prepared, for example, by adding 10 mL of B27, 5 mL of N2 Supplement (Thermo Fisher Scientific Inc.), and 1.6 mL of insulin at a concentration of 6.25 mg/mL to 500 mL of DMEMF12.

The culture medium used during, before, and after the infection with Sendai virus may contain B 18R proteins. B 18R proteins alleviate the innate antiviral response of the cells. B18R proteins may be used to inhibit cell-death associated with immune reactions associated with viral infection. However, since the method according to the embodiments is capable of producing nerve cell from the stem cells in a short period of time, the culture medium may not contain B18R proteins, or may contain B18R proteins in a low concentration of such as 0.01% to 1%.

The stem cells are induced into nerve cells within 25 days, 20 days, 10 days, 9 days, 8 days, 7 days, 6 days, 5 days, or 4 days from the infection with Sendai virus. Whether or not the stem cells are induced to nerve cells is, for example, confirmed by the positivity or negativity of one or more selected from MAP2, TUJ1 (13-III Tubulin), BRN2, HOMER1, NGN such as NGN2, PSA-NCAM, MUNC13-1, SATB2, vGLUT, chAT, HB9, LHX3, GAD65, GAD67, TH, ASCL such as ASCL1 (MASH1), vGAT, SOX1, SOX2, CD133, Nestin, HB9, ISL1, 04, PLP1, MOG, and MBP.

NGN2 is a switching protein required for producing neurons and is a marker for excitatory neurons. TUJ1 (13-III Tubulin), MAP2, BRN2, ASCL1 (MASH1), and PSA-NCAM are markers for neurons. HOMER1 and MUNC 13-1 are markers for mature neurons which form synapses. SATB2 is a marker for cerebrocortical neurons. vGLUT is a marker for excitatory neurons such as glutamatergic neurons. chAT is a marker for locomotor neurons such as cholinergic neurons. HB9 is a marker for locomotor neurons. GAD65 and GAD67 are markers for inhibitory neurons such as GABAergic neurons. TH is a marker for dopamine-producing neurons. vGAT is a marker for inhibitory neurons. SOX1, SOX2, CD133 and Nestin are markers for neural stem cells. LHX3, HB9, and ISL1 are markers for locomotor neurons. 04, PLP1, MOG, and MBP are markers for oligodendrocyte precursors. GFAP and CD44 are also available as markers for astrocyte precursors and astrocytes.

According to the method of the embodiments of the invention described above, it is possible to efficiently produce nerve cells integration-free and without damaging the genes of the stem cells by allowing the stem cells to express the mRNAs synthesizing the inducing factors.

In a method for producing nerve cells from stem cells using only hormones and chemicals, it requires quite a long time so that nerve cells are produced. Whereas, in the nerve cell production method according to the embodiments of the invention, it is possible to produce nerve cells in a short period of time.

Moreover, in the method for producing nerve cells from stem cells using only hormones and chemicals, only a portion of the stem cells become the intended nerve cells. Whereas, in the nerve cell production method according to the embodiments of the invention, most of the cells in which infection with Sendai virus has been established can become the intended nerve cells.

Furthermore, in the method for producing nerve cells from stem cells using only hormones and chemicals, there are clones that become the intended nerve cells and clones that do not although through the same protocol, and variation occurs among the clones. Whereas, in the method according to the embodiments of the invention, it is possible to obtain a high induction efficiency in a plurality of clones.

Moreover, when producing cells for transplantation through induction using cytokines from an undifferentiated cell population, there is a possibility that some uninduced cells remain in the cells for transplantation. Such remaining uninduced cells may independently expand through cell division at the transplantation site and form teratomas or the like. Whereas, in the nerve cell production method according to the embodiments of the invention, it is possible to allow simultaneous expression of mRNAs of drug resistance genes so that a drug selection of the cells into which Sendai virus has been introduced can be performed. Therefore, it is possible to avoid the risks such as contamination of uninduced cells and teratoma formation, and it is suitable for transplantation medicine.

Furthermore, in the method according to the embodiments of the invention, Sendai viruses are used and viruses which insert genes into the genomes are not used. Therefore, it is integration-free, does not damage the stem cell genes, and has no risks of cancerization in the produced nerve cells, making them available for clinical use.

Still further, cleaner and safer nerve cells can be produced by, for example, producing iPS cells from blood cells in a clean environment of a fully closed system, and subsequently producing the nerve cells from the iPS cells in a clean environment of a fully closed system by the method according to the embodiments of the invention.

Additionally, in the method according to the embodiments of the invention, since the nerve cells can be produced in a short period of time, it is not necessary to use B 18R or the like, and it can be set at a very low concentration even in case of use.

### EXAMPLE 1

A Sendai virus capable of expressing NGN2-Puro mRNA was prepared at ID Pharma Co., Ltd. The titer of the prepared Sendai virus was 1×10⁹ CIU/mL.

A 12-well dish coated with solubilized basement membrane preparation (Matrigel, Corning) was prepared, and a feeder-free culture medium (mTeSR^{R}1, Stemcell Technologies) containing ROCK (Rho-associated coiled-coil forming kinase/Rho-associated kinase) inhibitor (Selleck) at a concentration of 10 nmol/mL was added to each well. ROCK inhibitors inhibit cell death.

iPS cells were dispersed in detachment/dissociation/dispersion solution of tissues and cultured cells (Accutase, Innovative Cell Technologies) and seeded onto the 12-well dish. The iPS cells which are to be infected with Sendai virus were seeded at a density of 2×10⁵ cells/well. Control iPS cells which are not to be infected with Sendai virus were also seeded at a density of 2×10⁵ cells/well. Then, the iPS cells were cultured in feeder-free culture medium under the gas condition of 5% carbon dioxide concentration and 20% oxygen concentration for 24 hours.

The iPS cells were infected with Sendai virus capable of expressing NGN2-Puro mRNAs at a MOI of 20. On Day 2 after infection with Sendai virus, the culture medium in the wells was replaced with a neural induction culture medium (N3 culture medium) containing puromycin at a concentration of 2 μg/mL to kill the uninfected cells. The N3 culture medium has been prepared by adding 10 mL of B27, 5 mL of N2, and 1.6 mL of insulin with a concentration of 6.25 mg/mL to 500 mL of DMEMF12.

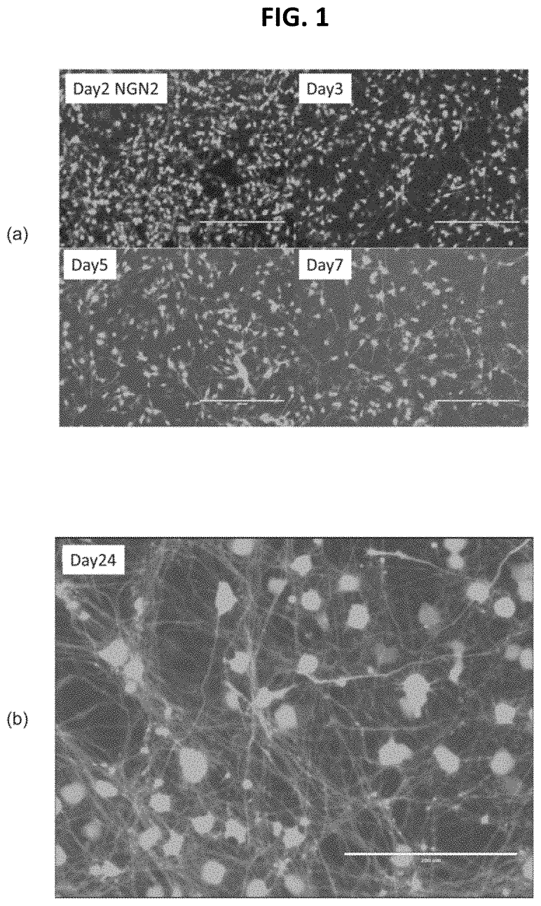

The microscope photographs of the cells on Day 2, Day 3, Day 5, and Day 7 after infection with Sendai virus are shown in FIG. 1(a). The microscope photograph of the cells on Day 24 after infection with Sendai virus is shown in FIG. 1(b). As shown in FIG. 1(a), induction of the cells to nerve cells after infection with Sendai virus has been morphologically confirmed.

On Day 28 after infection with Sendai virus, the culture medium was removed from the plate, and the cells were washed with PBS. Then, 4% paraformaldehyde (PFA) was added in the plate, and allowed to react for 15 minutes at 4° C. to fix the cells. The cells were further washed twice with PBS, and primary antibodies diluted with PBS culture medium containing 5% CCS and 0.1% Triton are added to the plate. As the primary antibodies, goat monoclonal antibodies (Santa Cruz Biotechnology) against BRN2 which is a marker for neurons were

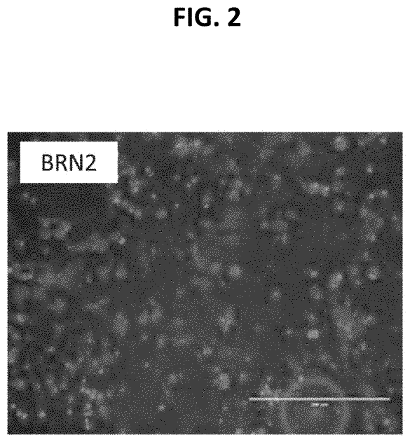

After allowing to react for lhour at room temperature, PBS was added to the plate, thoroughly mixed, and then the PBS was discarded. PBS was again added and discarded, and a solution containing a fluorescent-labeled gorilla anti-goat IgG(H+L) secondary antibodies (Alexa Fluor^{R}, 555, conjugate, ThermoFisher) was added to the plate, and allowed to react for 30 minutes at room temperature. The cells were then washed twice with PBS and observed under a fluorescence microscope. As a result, it has been confirmed that the induced nerve cells expressed BRN2 as shown in FIG. 2.

### EXAMPLE 2

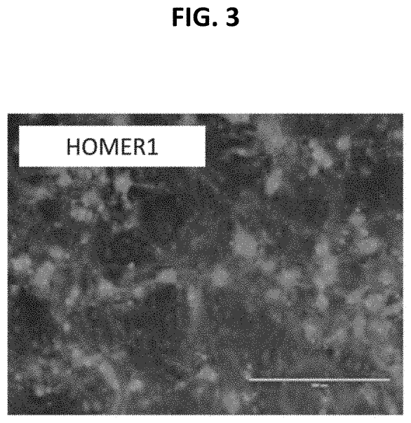

In the same manner as in Example 1, iPS cells were infected with Sendai virus capable of expressing NGN2-Puro mRNAs, the cells were fluorescent-labeled using rabbit monoclonal antibodies (SYNAPTIC SYSTEMS) against HOMER1 which is a marker for mature neurons forming synapses as primary antibodies, and observed under a fluorescence microscope. As a result, it has been confirmed that the induced nerve cells expressed HOMER1 as shown in FIG. 3.

### EXAMPLE 3

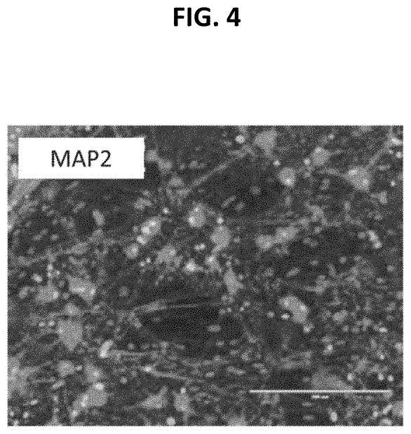

In the same manner as in Example 1, iPS cells were infected with Sendai virus capable of expressing NGN2-Puro mRNAs, the cells were fluorescent-labeled using mouse monoclonal antibodies (Sigma Aldrich) against MAP2 which is a marker for neurons as primary antibodies, and observed under a fluorescence microscope. As a result, it has been confirmed that the induced nerve cells expressed MAP2 as shown in FIG. 4.

### EXAMPLE 4

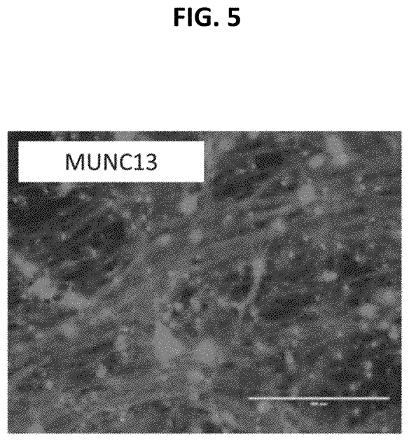

In the same manner as in Example 1, iPS cells were infected with Sendai virus capable of expressing NGN2-Puro mRNAs, the cells were fluorescent-labeled using rabbit monoclonal antibodies (SYNAPTIC SYSTEMS) against MUNC13-1 which is a marker for mature neurons forming synapses as primary antibodies, and observed under a fluorescence microscope. As a result, it has been confirmed that the induced nerve cells expressed MUNC13-1 as shown in FIG. 5.

### EXAMPLE 5

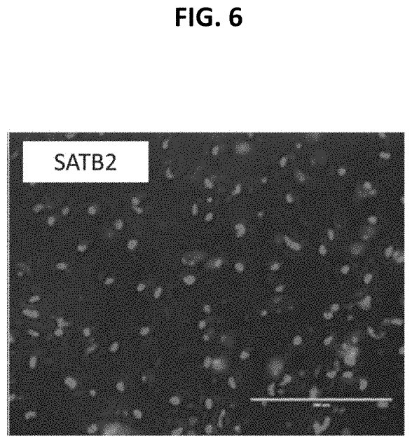

In the same manner as in Example 1, iPS cells were infected with Sendai virus capable of expressing NGN2-Puro mRNAs, the cells were fluorescent-labeled using mouse monoclonal antibodies (Abcam) against SATB2 which is a marker for cerebral neurons as primary antibodies, and observed under a fluorescence microscope. As a result, it has been confirmed that the induced nerve cells expressed SATB2 as shown in FIG. 6.

### EXAMPLE 6

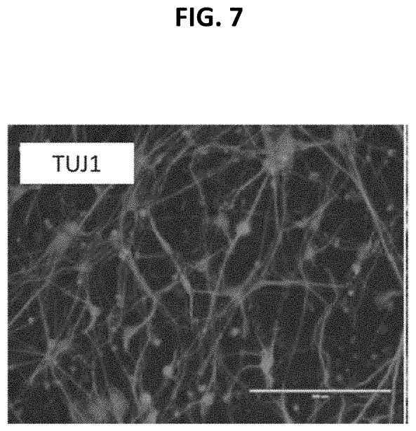

In the same manner as in Example 1, iPS cells were infected with Sendai virus capable of expressing NGN2-Puro mRNAs, the cells were fluorescent-labeled using mouse monoclonal antibodies (Biolegend) against TUJ1 which is a marker for neurons as primary antibodies, and observed under a fluorescence microscope. As a result, it has been confirmed that the induced nerve cells expressed TUJ1 as shown in FIG. 7.

### EXAMPLE 7

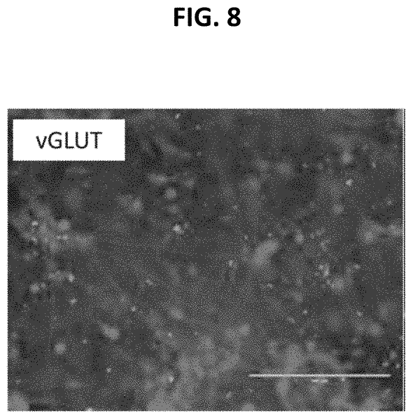

In the same manner as in Example 1, iPS cells were infected with Sendai virus capable of expressing NGN2-Puro mRNAs, the cells were fluorescent-labeled using rabbit monoclonal antibodies (SYNAPTIC SYSTEMS) against vGLUT which is a marker for excitatory neurons such as glutamatergic neurons as primary antibodies, and observed under a fluorescence microscope. As a result, it has been confirmed that the induced nerve cells expressed vGLUT as shown in FIG. 8.

### EXAMPLE 8

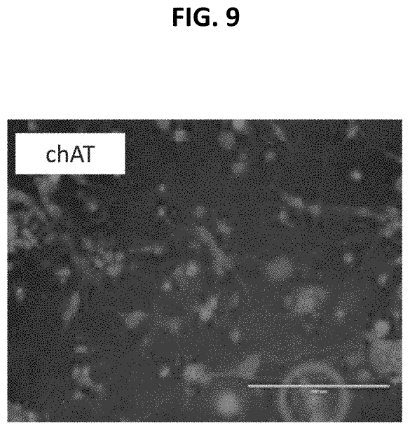

In the same manner as in Example 1, iPS cells were infected with Sendai virus capable of expressing NGN2-Puro mRNAs, the cells were fluorescent-labeled using mouse monoclonal antibodies (Millipore) against chAT which is a marker for locomotor neurons as primary antibodies, and observed under a fluorescence microscope. As a result, it has been confirmed that the induced nerve cells expressed chAT as shown in FIG. 9.

### EXAMPLE 9

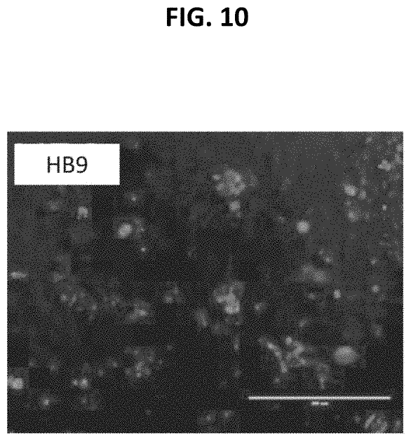

In the same manner as in Example 1, iPS cells were infected with Sendai virus capable of expressing NGN2-Puro mRNAs, the cells were fluorescent-labeled using mouse monoclonal antibodies (Thermo) against HB9 which is a marker for locomotor neurons as primary antibodies, and observed under a fluorescence microscope. As a result, it has been confirmed that the induced nerve cells expressed HB9 as shown in FIG. 10.

### EXAMPLE 10

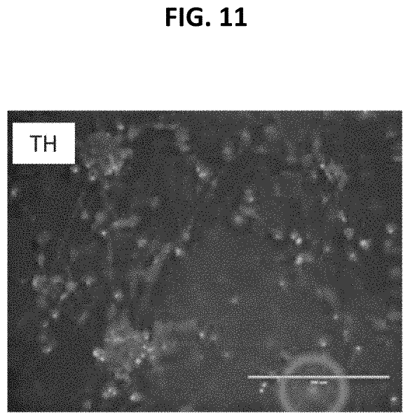

In the same manner as in Example 1, iPS cells were infected with Sendai virus capable of expressing NGN2-Puro mRNAs, the cells were fluorescent-labeled using sheep monoclonal antibodies (Pel Freez) against TH which is a marker for dopamine-producing neurons as primary antibodies, and observed under a fluorescence microscope. As a result, it has been confirmed that the induced nerve cells expressed TH as shown in FIG. 11.

### EXAMPLE 11

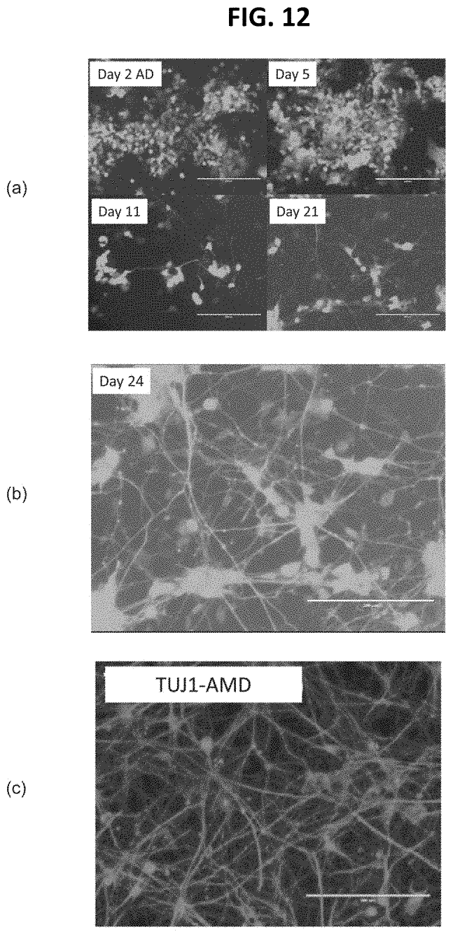

iPS cells were infected in the same manner as in Example 1 except that Sendai virus capable of expressing ASCL1-MYT1L-Puro mRNAs and DLX2-Blast mRNAs are used. The microscope photographs of the cells on Day 2, Day 5, Day 11, and Day 21 after infection with Sendai virus are shown in FIG. 12(a). The microscope photograph of the cells on Day 24 after infection with Sendai virus is shown in FIG. 12(b). As shown in FIG. 12(a) and FIG. 12(b), induction of the cells to nerve cells after infection with Sendai virus has been morphologically confirmed.

On Day 28 after infection with Sendai virus, the cells were fluorescent-labeled using mouse monoclonal antibodies (Biolegend) against TUJ1 which is a marker for average neurons as primary antibodies, and observed under a fluorescence microscope. As a result, it has been confirmed that the induced nerve cells expressed TUJ1 as shown in FIG. 12(c).

### EXAMPLE 12

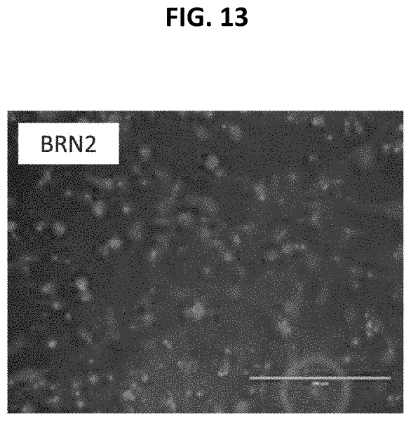

In the same manner as in Example 11, iPS cells were infected with Sendai virus capable of expressing ASCL1-MYT1L-Puro mRNAs and DLX2-Blast mRNAs, the cells were fluorescent-labeled using goat monoclonal antibodies (Santa Cruz Biotechnology) against BRN2 which is a marker for neurons as primary antibodies, and observed under a fluorescence microscope. As a result, it has been confirmed that the induced nerve cells expressed BRN2 as shown in FIG. 13.

### EXAMPLE 13

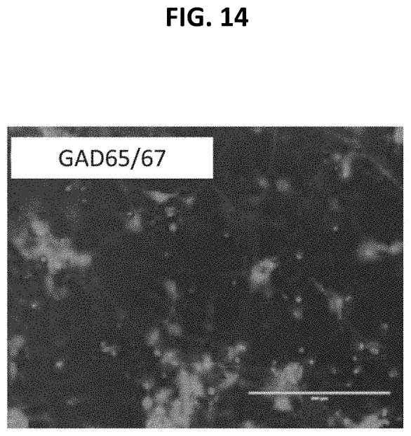

In the same manner as in Example 11, iPS cells were infected with Sendai virus capable of expressing ASCL1-MYT1L-Puro mRNAs and DLX2-Blast mRNAs, the cells were fluorescent-labeled using mouse monoclonal antibodies (Millipore) against GAD65/67 which are markers for inhibitory (GABAergic) neurons as primary antibodies, and observed under a fluorescence microscope. As a result, it has been confirmed that the induced nerve cells expressed GAD65/67 as shown in FIG. 14.

### EXAMPLE 14

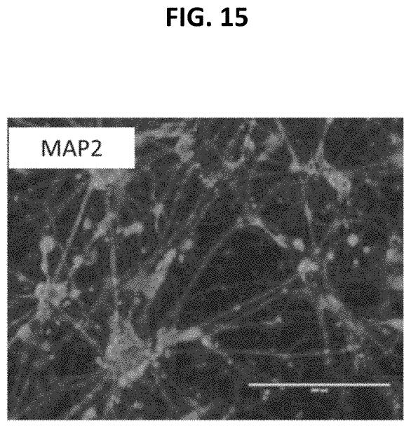

In the same manner as in Example 11, iPS cells were infected with Sendai virus capable of expressing ASCL1-MYT1L-Puro mRNAs and DLX2-Blast mRNAs, the cells were fluorescent-labeled using mouse monoclonal antibodies (Sigma Aldrich) against MAP2 which is a marker for neurons as primary antibodies, and observed under a fluorescence microscope. As a result, it has been confirmed that the induced nerve cells expressed MAP2 as shown in FIG. 15.

### EXAMPLE 15

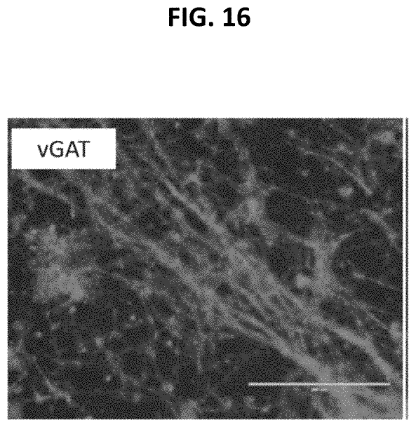

In the same manner as in Example 11, iPS cells were infected with Sendai virus capable of expressing ASCL1-MYT1L-Puro mRNAs and DLX2-Blast mRNAs, the cells were fluorescent-labeled using rabbit monoclonal antibodies (SYNAPTIC SYSTEMS) against vGAT which is a marker for inhibitory neurons as primary antibodies, and observed under a fluorescence microscope. As a result, it has been confirmed that the induced nerve cells expressed vGAT as shown in FIG. 16.

### EXAMPLE 16

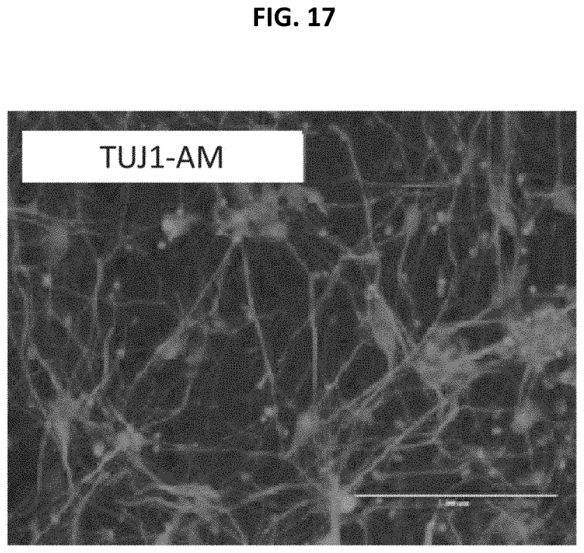

iPS cells were infected in the same manner as in Example 1 except that Sendai virus capable of expressing ASCL1-MYT1L-Puro mRNAs are used. On Day 21 after infection with Sendai virus, the cells were fluorescent-labeled using mouse monoclonal antibodies (Biolegend) against TUJ1 which is a marker for average neurons as primary antibodies, and observed under a fluorescence microscope. As a result, it has been confirmed that the induced nerve cells expressed TUJ1 as shown in FIG. 17.

### EXAMPLE 17

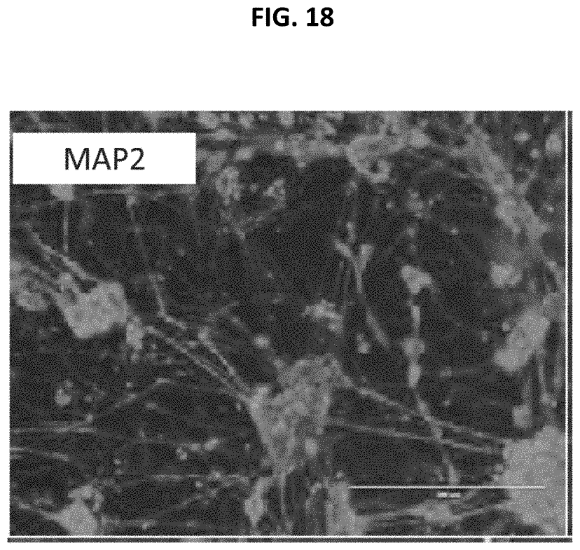

In the same manner as in Example 22, iPS cells were infected with Sendai virus capable of expressing ASCL1-MYT1L-Puro mRNAs, the cells were fluorescent-labeled using mouse monoclonal antibodies (Sigma Aldrich) against MAP2 which is a marker for neurons as primary antibodies, and observed under a fluorescence microscope. As a result, it has been confirmed that the induced nerve cells expressed MAP2 as shown in FIG. 18.

### EXAMPLE 18

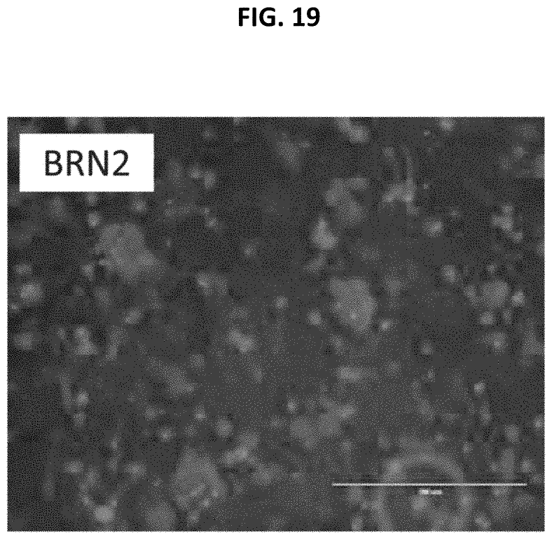

In the same manner as in Example 22, iPS cells were infected with Sendai virus capable of expressing ASCL1-MYT1L-Puro mRNAs, the cells were fluorescent-labeled using goat monoclonal antibodies (Santa Cruz Biotechnology) against BRN2 which is a marker for neurons as primary antibodies, and observed under a fluorescence microscope. As a result, it has been confirmed that the induced nerve cells expressed BRN2 as shown in FIG. 19.

### EXAMPLE 19

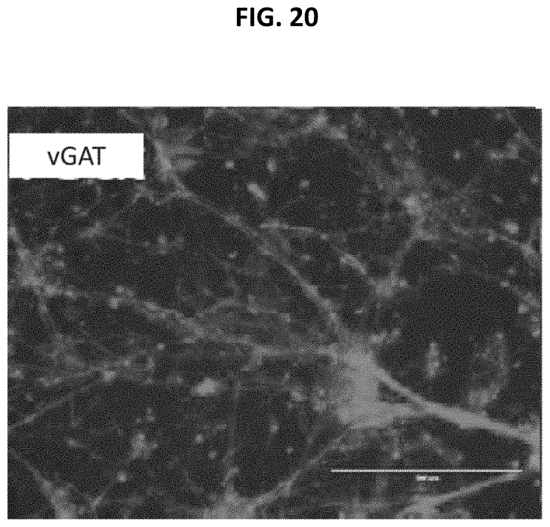

In the same manner as in Example 22, iPS cells were infected with Sendai virus capable of expressing ASCL1-MYT1L-Puro mRNAs, the cells were fluorescent-labeled using rabbit monoclonal antibodies (SYNAPTIC SYSTEMS) against vGAT which is a marker for inhibitory neurons as primary antibodies, and observed under a fluorescence microscope. As a result, it has been confirmed that the induced nerve cells expressed vGAT as shown in FIG. 20.

### EXAMPLE 20

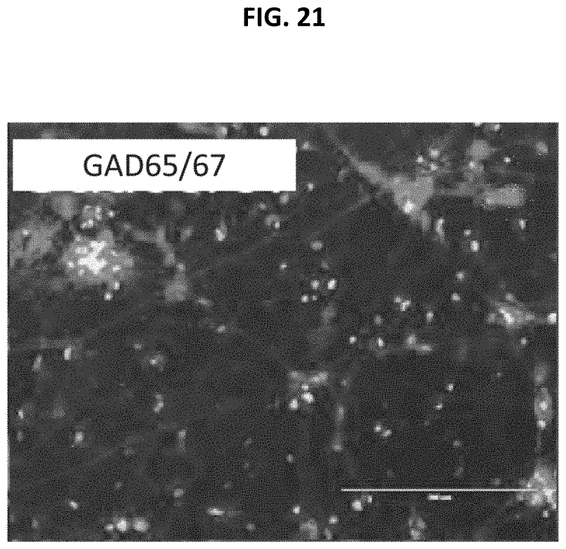

In the same manner as in Example 22, iPS cells were infected with Sendai virus capable of expressing ASCL1-MYT1L-Puro mRNAs, the cells were fluorescent-labeled using mouse monoclonal antibodies (Millipore) against GAD65/67 which are markers for inhibitory neurons (GABAergic neurons) as primary antibodies, and observed under a fluorescence microscope. As a result, it has been confirmed that the induced nerve cells expressed GAD65/67 as shown in FIG. 21.

### EXAMPLE 21

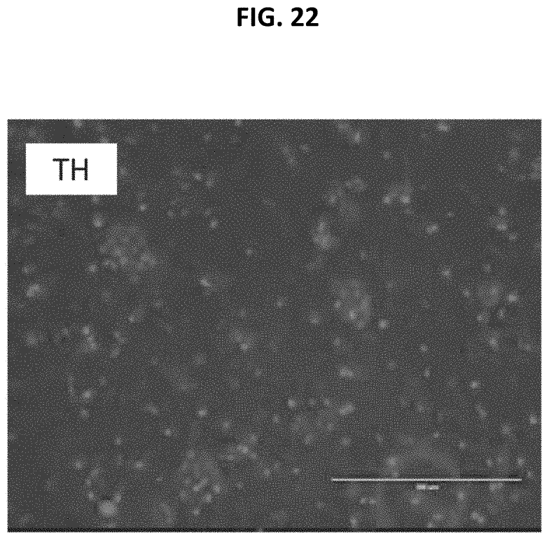

In the same manner as in Example 22, iPS cells were infected with Sendai virus capable of expressing ASCL1-MYT1L-Puro mRNAs, the cells were fluorescent-labeled using sheep monoclonal antibodies (Pel Freez) against TH which is a marker for dopamine-producing neurons as primary antibodies, and observed under a fluorescence microscope. As a result, it has been confirmed that the induced nerve cells expressed TH as shown in FIG. 22.

### EXAMPLE 22

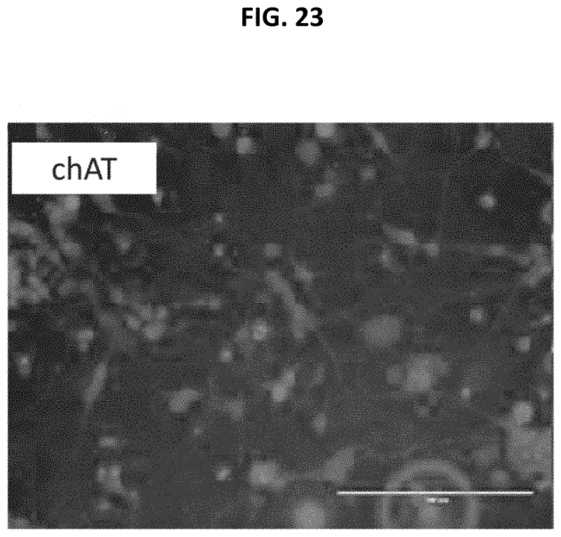

In the same manner as in Example 22, iPS cells were infected with Sendai virus capable of expressing ASCL1-MYT1L-Puro mRNAs, the cells were fluorescent-labeled using goat monoclonal antibodies (Millipore) against chAT which is a marker for cholinergic neurons as primary antibodies, and observed under a fluorescence microscope. As a result, it has been confirmed that the induced nerve cells expressed chAT as shown in FIG. 23.

### EXAMPLE 23

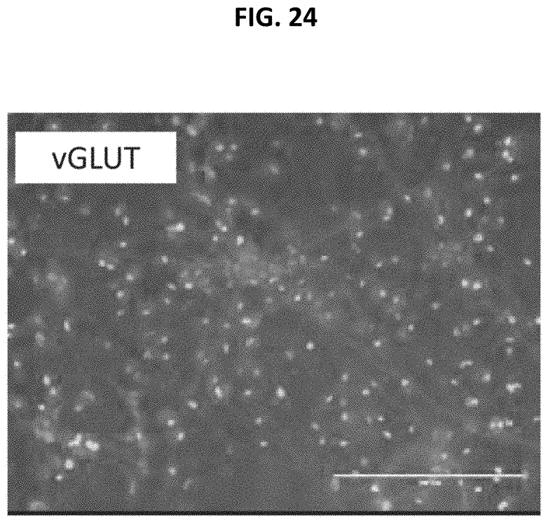

In the same manner as in Example 22, iPS cells were infected with Sendai virus capable of expressing ASCL1-MYT1L-Puro mRNAs, the cells were fluorescent-labeled using rabbit monoclonal antibodies (SYNAPTIC SYSTEMS) against vGLUT which is a marker for excitatory neurons (glutamatergic neurons) as primary antibodies, and observed under a fluorescence microscope. As a result, it has been confirmed that the induced nerve cells expressed vGLUT as shown in FIG. 24.

### EXAMPLE 24

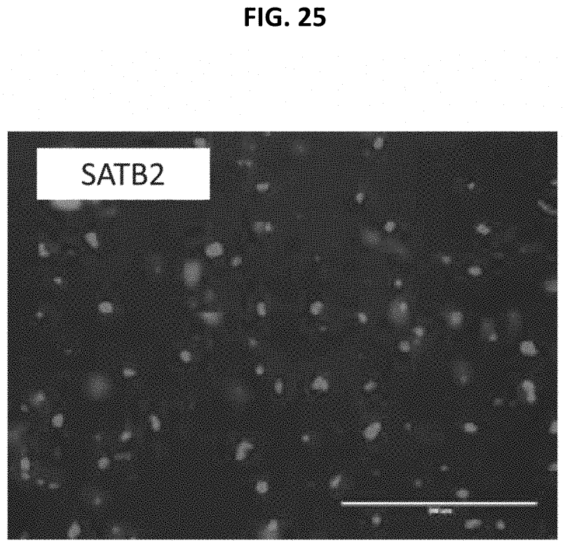

In the same manner as in Example 22, iPS cells were infected with Sendai virus capable of expressing ASCL1-MYT1L-Puro mRNAs, the cells were fluorescent-labeled using mouse monoclonal antibodies (Abcam) against SATB2 which is a marker for cerebral neurons as primary antibodies, and observed under a fluorescence microscope. As a result, it has been confirmed that the induced nerve cells expressed SATB2 as shown in FIG. 25.

### EXAMPLE 25

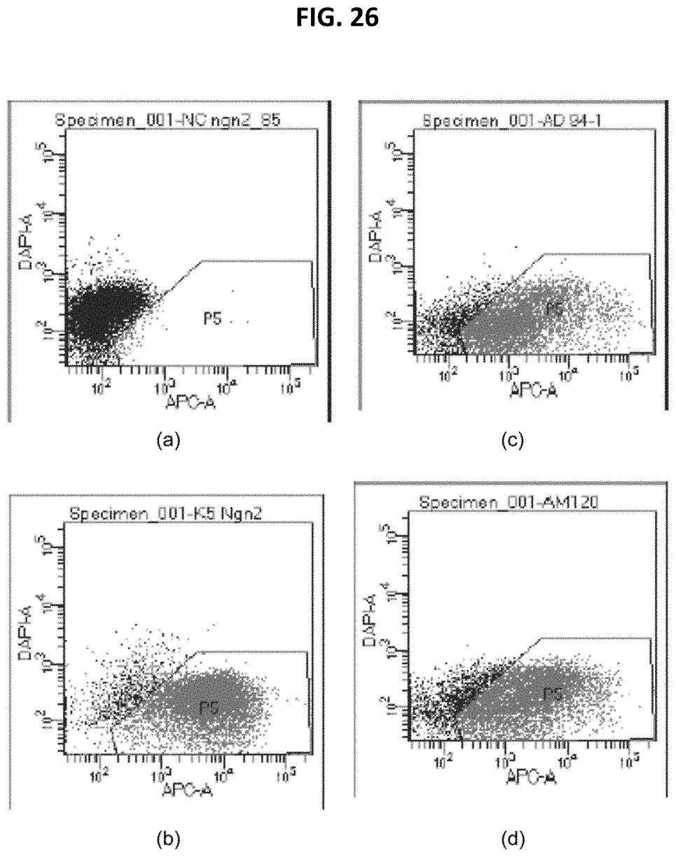

A flow cytometry analysis regarding the control cells which had not been infected with Sendai virus capable of expressing NGN2-Puro mRNAs prepared in Example 1 showed that the control cells were negative for PSA-NCAM which is a marker for neurons, as shown in FIG. 26(a). A flow cytometry analysis on Day 7 after the infection regarding the cells which had been infected with Sendai virus capable of expressing NGN2-Puro mRNAs prepared in Example 1 showed that the cells were positive to PSA-NCAM which is a marker for neurons, as shown in FIG. 26(b). Incidentally, the cells were also positive to PSA-NCAM which is a marker for neurons on a flow cytometry analysis on Day 4 after infection.

A flow cytometry analysis on Day 7 after the infection regarding the cells which had been infected with Sendai virus capable of expressing ASCL1-MYT1L-Puro mRNAs and DLX2-Blast mRNAs prepared in Example 11 showed that the cells were positive to PSA-NCAM which is a marker for neurons, as shown in FIG. 26(c). Incidentally, the cells were also positive to PSA-NCAM which is a marker for neurons on a flow cytometry analysis on Day 4 after infection. A flow cytometry analysis on Day 7 after the infection regarding the cells which had been infected with Sendai virus capable of expressing, ASCL1-MYT1L-Puro mRNAs prepared in Example 22 showed that the cells were positive to PSA-NCAM which is a marker for neurons, as shown in FIG. 26(d). Incidentally, the cells were also positive to PSA-NCAM which is a marker for neurons on a flow cytometry analysis on Day 4 after infection.

1. An excitatory neuron production method comprising:
preparing human stem cells, and
introducing Sendai virus by infection to the human stem cells in a culture medium which does not contain B 18R protein or which contains 0.01% to 1% B 18R protein to induce the human stem cells into excitatory neurons by allowing the Sendai virus to express mRNA which synthesizes an inducing factor including neurogenin (NGN), or an inducing factor including achaete-scute homolog (ASCL) and myelin transcription factor (MYT) in the human stem cells.

2. The excitatory neuron production method according to claim 1, wherein the human stem cells are induced pluripotent stem cells.

3.-8. (canceled)

9. The excitatory neuron production method according to claim 1, wherein the excitatory neurons are positive for TH, TUJ1, BRN2, NGN, β-III Tubulin, MAP2, PSA-NCAM, and vGLUT, SATB2, chAT, HB9, MUNC13, HOMER1.

10. The excitatory neuron production method according to claim 1, wherein the Sendai virus is allowed to express mRNA of a drug resistance gene in the human stem cells.

11.-12. (canceled)

13. An inhibitory neuron production method comprising:
preparing stem cells, and
introducing Sendai virus by infection to the stem cells in a culture medium which does not contain B 18R protein or which contains 0.01% to 1% B 18R protein to induce the stem cells into inhibitory neurons by allowing the Sendai virus to express mRNA which synthesizes an inducing factor including achaete-scute homolog (ASCL) in the stem cells.

14. The inhibitory neuron production method according to claim 13, wherein the stem cells are induced pluripotent stem cells.

15. The inhibitory neuron production method according to claim 13, wherein the stem cells are embryonic stem cells.

16. The inhibitory neuron production method according to claim 13, wherein the inducing factor further includes myelin transcription factor (MYT).

17. The inhibitory neuron production method according to claim 13, wherein the inducing factor further includes distal-less homeobox (DLX).

18. The inhibitory neuron production method according to claim 13, wherein the inhibitory neurons are positive for one or more selected from the group consisting of vGAT and GAD65/67.

19. The inhibitory neuron production method according to claim 13, wherein the Sendai virus is allowed to express mRNA of a drug resistance gene in the stem cells.

20. (canceled)

21. A cerebral neuron production method comprising:
preparing stem cells, and
introducing Sendai virus by infection to the stem cells to induce the stem cells into cerebral neurons by allowing the Sendai virus to express mRNA which synthesizes an inducing factor including neurogenin (NGN), or an inducing factor including achaete-scute homolog (ASCL) and myelin transcription factor (MYT) in the stem cells.

22.-23. (canceled)

24. The cerebral neuron production method according to claim 21, wherein the cerebral neurons are positive for SATB2.

25. The cerebral neuron production method according to claim 21, wherein the Sendai virus is allowed to express mRNA of a drug resistance gene in the stem cells.

26. (canceled)

27. A synapse-forming neuron production method comprising:
preparing stem cells, and
introducing Sendai virus by infection to the stem cells to induce the stem cells into synapse-forming neurons by allowing the Sendai virus to express mRNA which synthesizes an inducing factor including neurogenin (NGN) in the stem cells.

28.-29. (canceled)

30. The synapse-forming neuron production method according to claim 27, wherein the synapse-forming neurons are positive for one or more selected from the group consisting of HOMER1 and MUNC13-1.

31. The synapse-forming neuron production method according to claim 27, wherein the Sendai virus is allowed to express mRNA of a drug resistance gene in the stem cells.

32. (canceled)

33. A dopamine-producing neuron production method comprising:
preparing stem cells, and
introducing Sendai virus by infection to the stem cells to induce the stem cells into dopamine-producing neurons by allowing the Sendai virus to express mRNA which synthesizes an inducing factor including neurogenin (NGN), or an inducing factor including achaete-scute homolog (ASCL) and myelin transcription factor (MYT) in the stem cells.

34.- 35. (canceled)

36. The dopamine-producing neuron production method according to claim 33, wherein the dopamine-producing neurons are positive for TH.

37. The dopamine-producing neuron production method according to claim 33, wherein the Sendai virus is allowed to express mRNA of a drug resistance gene in the stem cells.

38. (canceled)


SEQUENCE LISTING (10 sequences)

SEQ ID NO: 1 - DNA, 2244 bp, Artificial Sequence
  Inducing factor
gcagccactgaaccacaagcagctcggctttaactggagtgccttggagtcgcgtgccagcagccacacggccagggactgactgacagacaaccacgcacgagaacgacaacacacgagactcgggcgagctgccgcggtcgtccgggctcttggcaaagtcgcccagccgagaggcccccccgcggaggtgcgcctaggaagcgccaagcccgcggcgcggaggacaccgtgctcggttccgggctgcggggacattcccggacacacaccggagcagcagctgcgccgcgacacatctggagccgcgtaggatgttcgtcaaatctgagactctggagttgaaggaggaagaggaggtactgatgctgctgggctcggcttccccggcctcggcgaccctgaccccgatgtcctccagcgcggacgaggaggaggacgaggagctgcgccggccgggctccgcgcgtgggcagcgtggagcggaagccgggcagggggtgcagggcagtccggcgtcgggtgccgggggttgccggccagggcggctgctgggcctgatgcacgagtgcaagcgtcgcccgtcgcgctcacgggccgtctcccgaggtgccaagacggcggagacggtgcagcgcatcaagaagacccgcaggctcaaggccaacaaccgcgagcgcaaccgcatgcacaacctaaacgccgcgctggacgcgctgcgcgaggtgctgcccaccttccccgaggatgccaagctcacgaagatcgagacgctgcgcttcgcccacaattacatctgggcgctcaccgagactctgcgcctggcggaccactgcgccggcgccggtggcctccagggggcgctcttcacggaggcggtgctcctgagcccgggagctgcgctcggcgccagcggggacagcccttctccaccttcctcctggagctgcaccaacagcccggcgtcatcctccaactccacgtccccatacagctgcactttatcgcccgctagccccgggtcagacgtggactactggcagcccccacctccggagaagcatcgttatgcgcctcacctgcccctcgccagggactgtatctagagctgcgggtctccctctctcgtcctctacccggccctcttcccatccttctcccgcccctcaccctccacgccccggactccacttcacagagcagaggtggcccttgcaatcccctcggcggctggtgcattcgggggtggagaccagctctggtttattgaagatgtgaggatttatggtcaaagaggactatggcgtgtgggagtgggggctggcgtggggaacctcgtaagactgtaaaagacactgagaaaaagtaccataactaacgagtgtgcagagcagactgacgctcctcccctctctcagagctgctggaggagaactccgggcaggcagttcgtgtgaatctctcagagggaatgcaactggtccctgtgatcttttcaccttcgtttctacatagagatgttaatgtcagtcgaaagaaatgtattttagcatctgaatgaatttactggtaataatattatccacacatttgcaatggctggcatctgctctattcccattgctgtctgcaggctgtgggaatttcacctgtcaaaccaaactttccctctctgatgtgcactttgtttttttcccagattcgtcacaatgcctattgtcccgcccttctttttgctttttttctccattttgccatctgtctcttatgatttataagggggaaaaacttgttttgttagagggccaggttagaagtcattgtataatttgtaggcttttgtaagggttgaatgcaagcgtggaaatttaggctgaattctctatcaaaagaaaaaatgtgaaggaaaaaggaaaaatcaggagggaggattgcttcatgcattatttatctcgaccttttaggggagaaggaactcccccatcctttcaagagattaaaaataaatcaacagtctgaaaacctaagcagacacggggcattgccaggatcagccacacacgtgtttccttctatttattttgaagaaaaatttcatgggaaagtatgtatttttttgtatattctacagagtttattctagtatgtatttacatcccgaagaataagaaaattgttttgtgattaagctataaataaagtatctaattttcataaaaaaaaaaaaaaaaaaaaaaaaaaaaa

SEQ ID NO: 2 - DNA, 2370 bp, Artificial Sequence
  Inducing factor
cgcagccactgaaccacaagcagcttcgcgttaactggagtgcctgggagtcgcgtgccaggagccgcacggccagggactgactgacagacagacacgcaccaccaccacaacacacgagacccgggcgggccgccgccgccgccgccggggctcttggcaaactcgccggtcgcagaggtcccccgcggagctgcgccacagtagcgccgggcttgcagctttcacgccgggcgaaggacccggcgctgcgctcgcagctgcgcggagattcccggcacaggccaaagtcacagcaacgctgaggcacagttagagccaactaagatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggagccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctagagctgccatttctgctacccacgccaggccttagtgggttccctttcctgtccccagtcgagccctcctcccttcccctgcccctcctttccacgccctggaaaccatctcacttcacagggcaggtgtagcctttctgattcctcggttgtttcttgcatttcttggctttgggtatccttcattcagacgggctctgatttactgaaggtgtgatggagcttattgtcaaagccaagggtggcgttttgggggcgcttcttgagacgaaaaagaccctgggaagagatgatggtggcatatctaaagagtttgcagagcggactgacgctcctcccctttctctttaacgccgaaggacttggtgcagttcgtgtgaatctcacagggggaatgcaactggttcctgtgatctcttcacctttgcttctacatagagatgttaatgtcgagtagaaagaaatgtatcttagcatctgaatgattttgctggtaataatattatccacagatttgcaatggctggcatctgctttattcccattgctgtctgcaggctgtgggaatttcacctgtcaaaccaaacttccctctctgatgtgcactttgttctgtttcccagattcgtcacaatgcctattgtcctgtccttctctttcctttttcttccccattttgccatctgtctcttatgatttataaggggaaaaaaacttgttttgttagaggggcaggttagaagtcattgtataatttgtaggctttgtaatgattgaatgcaagcgtggaaatttaggctgaactctctatcaaaaggaaaaatgtggaggaaaagggaaaaatcaggagggaggattgcctcatgtattatttatttcgaccttttaggggagaaggaactcccccattctttcaagagattaaaaataaatcaacagtctgaaaacctaagcagacacggagcattatccggatcagccacacacgtgttcccttctatttattataaagaaatttttcatgggaaaatatgtattttttgtatattctacagagtttattctagtatgtatttacatcttgaagaacaagaaagttgttcttgtgattaaactataaataaactatctaattttcataaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 2259 bp, Artificial Sequence
  Inducing factor
agcagtctctcacttctggccagggaacgtggaaggcgtaccggctgggagccggttagggagggcgaattggggggaacgagagagcaattagaaagaaagggggttcaaccaaataatcccagaagcaggctcaagcccaggctggagcaagggagagcgggcgcaagaaagcgcagccccggagcagctccacctggcagagtgcgctcggcactgacttttgcggctgctttccttttccctttcctcttttaaaaccgagaaggcgccggcggcggccgcacacgcgagcgccacgcgaggctcccgaagccaaccgcggcgggaggaggggagggaggaggcggcgcagagggaagacgatcgcccaggcaccttcctccgctgcagcctgacaactctgcctccttctgcgcgtttcttcccctttaactttcctccggggctcgtttctcccctctcctttttcttcgtccccctttgatcgtgcttcgcagccccgcttccttcaagggctctgcgcaccctgcgtccccaactcgttctcccccgcgacagtttggcccggcatggagagctctggcaagatggagagtggagccggccagcagccgcagcccccgcagcccttcctgcctcccgcagcctgcttctttgcgaccgcggcggcggcggcagcggcggcggccgcggcagctcagagcgcgcagcagcaacagccgcaggcgccgccgcagcaggcgccgcagctgagcccggtggccgacagccagccctcagggggcggtcacaagtcagcggccaagcaggtcaagcgccagcgctcgtcctctccggaactgatgcgctgcaaacgccggctcaacttcagcggcttcggctacagcctgccacagcagcagccggccgccgtggcgcgccgcaacgagcgcgagcgcaaccgggtcaagttggtcaacctgggttttgccaccctccgggagcatgtccccaacggcgcggccaacaagaagatgagcaaggtggagacgctgcgctcggcggtcgagtacatccgcgcgctgcagcagctgctggacgagcacgacgcggtgagcgctgcctttcaggcgggcgtcctgtcgcccaccatctcccccaactactccaacgacttgaactctatggcgggttctccggtctcgtcctactcctccgacgagggatcctacgaccctcttagcccagaggaacaagagctgctggactttaccaactggttctgaggacctgccaggctctcctgggaatggactttggaagcaggatggcagcagatcctgcatctttagtgtttctcgccaacgacgtcaaatggggaggcagaaaaacaaggggaaaaaagaagaagaaatgaaacaaacaaaccagacagccaacctacaggggcaccttcactaagatgcaatgttctcagcaaacaggggtgggctccaacagtgtctctgcattccaacatcatttccagacacgagaagagtgactggtgtctgaacctaagcccgaatcacagatgggttcctttcctggagcaagagcgtcacacacacacacacacacacagacagacactatattaactcccaaccactaacaggcagggctggaagcgcgcatgtgcaagtgccttcacctcccactctctgtcagagctgtcttagccccctgaaactgggttgatgtctttcctcagtcacccccattccagcgatctatggacatttgcctccattgaagcaacgtcagttctcggacagcctttccctctcctggtggcctcctccccaaaccccacatcgccctcccacggtctttgcttctgttttcttcatagaatgcttccaatctttgtgaatttttttattataagaaaaaaatctatttgtatctatcctaaccagtttggggatatattaagatatttttgtacataagaaaaagagagagaaaaaatttatagaagttttgtacaaatggtttaaaaatgtgtatatcttgatactttaacatgtaatgagattacctctgcgtactttagatatgtagttcatcttacaactgccatccccacccccatccccagtgtggttttggaaagaactctcctcataggtgagatctaaatgccaccagaatgacttcagcaccaatgtgtcttacttcacagaaacgtggttaatgtattaatgatgttattaaaaaaaactgttcaagaag

SEQ ID NO: 4 - DNA, 2490 bp, Artificial Sequence
  Inducing factor
agcactctctcacttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctcctcgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgcctttttttttcttactgttttattacaaacttacaaaaatatgtataaccctgttttatacaaactagtttcgtaataaaactttttcctttttttaaaatgaaaataaaaaaaa

SEQ ID NO: 5 - DNA, 7198 bp, Artificial Sequence
  Inducing factor
gtggagctgcgtgcattaggggctggctgtggaggtaaagcagatctcttaggcccgctagcctctcctgctgcatgtgtcattcctgcccgcgcgacattttccccctaagctactgcacacaaaacagagcgagaaagcttctccctgcagtcttcttggaggcctcctggtttctcacccattgttggtgggtgtatttcaattttttgattccctggactgtgggttatgaaatcaatgctgctgaagacaaaagcaaccttccctgcctctcggtgctatgcgtggtcctcctcggcctcccacttgtgggggaaagggttttctctttctttctgtgtgttttgagccagcacagttaccaaaattgaacttgccgtcacttgtgagcggtgtggtcatggtgtgaggggtcccacagaggctgcagctgaggtctgggtgtgtgcaattctcagctgggctttgccctacccaggttgacgactgaagaatcacagagtgtggaaaagaacacaaggagaaattggtgagaacatctgcctagcatctccaagtcctgtggagggagccagcagtgctggtccaaagaggacccagagagtgaactcagagtgaccacatctgatagaagaggggaagatgtagtttctgagtccagtccagtgtttgtgtctctcacattgtcaacaaaagaaaggctccagctgtccccacagacatatggatattccaggagccacgtaaagatggagaaatggaggcacagagaaattaagtgacttggccacagtcacaagctggggaggaccagggaaagcctagagagagctggctctgggcctgcatcctgcccacggagtcaccctgcctccgtcctcaggagagaaggcttcctacaagatggacgtggactctgaggagaagcgccatcgcacacggtccaaaggggttcgagttcctgtggagccagccatacaagagctgttcagctgtcccactccaggctgcgacggcagtggtcacgtcagtggcaaatatgcacgacacagaagtgtatatggttgtcccttggctaaaaaaagaaaaacgcaagataaacagccccaagaacctgctcccaagcgaaaaccatttgcagtaaaagcagatagttcctcagtagacgaatgttatgagagtgatggtactgaagacatggatgataaggaggaagatgatgatgaggagttctctgaagacaatgatgagcaaggggatgatgacgacgaagatgaggtggatcgggaagacgaggaggagatcgaggaggaagatgatgaagaagatgatgatgatgaagatggtgacgatgtagaagaggaagaagaggatgatgatgaagaggaggaagaagaggaagaggaagaagaaaatgaagaccatcaaatgagttgtactcgaataatgcaggacacagacaaggatgataacaacaatgatgagtatgataactatgatgaactggtagctaagtcgctattaaatcttggcaaaattgctgaggatgcagcataccgagccaggactgaatcagagatgaacagcaatacctccaatagtctggaggacgatagtgacaaaaacgaaaacctcggtcggaaaagcgaactgagtctagacttagacagtgatgttgttagagaaacagtggactcccttaagctgttagcacaaggacatggtgttgtgctatcagagaatatcagtgacagaagttatgctgaggggatgtcacagcaggacagtagaaatatgaactatgtcatgctagggaagcccatgaacaatggactcatggagaagatggtggaggagagtgatgaggaagtgtgtctaagtagtctagagtgcctgaggaaccagtgctttgacctggccaggaaactcagcgagaccaacccacaggacaggagtcagccacccaacatgagtgtgcgccaacatgtccggcaagaggacgacttccctgggaggacgccagacaggagctactcggatatgatgaaccttatgcggctggaggagcagctcagtcccaggtctagaacgttctccagctgtgccaaggaggatgggtgtcatgagagggatgatgacaccacctcagtgaactcagacaggtctgaggaagtgtttgacatgaccaagggcaacctgactctgctagagaaagccattgccttggagacagagagagccaaggccatgcgggagaagatggccatggatgctgggagaagggataacctgagatcctatgaggaccagtctccaagacagctggctggggaagacagaaaatccaaatccagtgacagccatgtcaaaaagccatactatggtaaagatccctcaagaacagaaaagagagagagcaagtgtccaacccccgggtgtgatggaaccggccacgtaactgggctttacccgcatcaccgcagtctgtctggatgcccgcacaaagatagggtccctccagaaattcttgccatgcatgaaaatgttctcaagtgtcccactccaggctgcacagggcgagggcatgtgaatagcaacaggaactcgcacagaagcctctctggatgccccattgctgctgcagaaaaactggcaaaggcccaagagaaacaccagagctgtgatgtgtccaaatccaaccaggcctcagaccgagtcctcaggccaatgtgctttgtcaaacagcttgagattcctcagtatggctacagaaacaatgttcccacaaccacaccacgctccaacctggccaaggagcttgagaaatactccaagacttcgtttgagtacaacagttacgacaaccatacttatggcaaaagagccatagctcccaaggtgcaaaccagggacatatcccccaaaggatatgacgatgccaagcggtactgcaagaatgccagccccagcagcagcaccaccagcagctatgcacctagcagcagcagcaacctcagctgtggtggtggcagcagcgccagtagcacgtgtagcaagagcagctttgactacacacatgacatggaggccgcacacatggcagccacagccattctcaacctgtccacacgttgtcgtgaaatgccacagaacctgtccaccaagccacaggacctgtgtactgcccggaacccagacatggaggtggatgagaatggcaccctggacctgagcatgaacaagcagaggcctcgagacagctgctgcccagtcctgacacccctggaacccatgtctccgcagcagcaggccgtgatgagcagccgatgcttccagctgagcgagggggattgctgggacttgcctgtagactacaccaaaatgaagcctcggagggtagatgaggatgagcccaaagagattaccccagaagacttggacccattccaggaggctctggaagaaagacggtatccaggggaggtgaccatcccaagccccaaacccaagtaccctcagtgcaaggaaagcaaaaaggacttaataactctgtctggctgccccctggcggacaaaagcattcgaagtatgctggccaccagttcccaagagctcaagtgccccacccctggctgtgacggttctggacacatcactggcaattacgcttctcatcgaagcctttctgggtgcccgagagcaaagaagagtggcatccggatagcacagagcaaagaggacaaggaagaccaggagccaatcaggtgtccggtacctggctgtgacggtcagggacacatcactgggaagtatgcatcccaccgcagcgcctccgggtgtcccttggcagccaagaggcagaaagatgggtaccttaatggctcccagttctcctggaagtcggtcaagacggagggcatgtcctgccctacccccgggtgtgatgggtcaggacacgtcagtggcagcttcctcacacaccgcagcttgtcaggatgtccaagagccacatcagcaatgaagaaagcaaagctgtctggagaacagatgttgactatcaagcagcgagccagcaacggtatagaaaatgatgaagaaatcaagcagttagatgaagagatcaaggagcttaatgagtccaattcccagatggaggctgacatgatcaaactcagaactcagatcaccacaatggagagcaacctgaagacgattgaggaggagaacaaagtcattgaacagcagaatgagtcgctcttgcacgagttggccaacctgagccagtccctgatccacagcctcgccaacatccagctgcctcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctgaacagctgctctagtggtgactcatgcttaaaaaggatgcctcttgtttcttgctgctgtaacttaccagaaagtgttatatatttatttctgtcggaacagtgttatgctacaagacttcataatggttttgtgtgctctcgagagagtacctgcagactagttttggatacattcacattttgtacgttttcatataagctgacatagtgtgatttgccatgtaatgtttatagctgctgctgtctgcacatttgggggtctctatatttctgaagaggtaagctgatgaaaataaatagagtgtaaattctttttaatgctttagtgattaaatgttttagtattttgaactgaaatggacaaaaaaagaaaaaggaaaaaaaaagcaggtttgaacgatcactttgtggcctcctggccacttttagacattatcattttgcctcaggttggaggactttgtggaatttaagaaatacattttgtgtgcatattgtttcatagcaagaattggttgcaaaaaaatgctttatttttgaacaatgcttggaaatattatgtgacttttttgtttgtttgttttaggaggatggtgtatggtgggggcaataaatgaggttttttgcattccaaggaaatggcatatggattaactataagaaatgaaataagtaatttattgtaagacaacatcaagccatggaaacttggcagaagattcaaagcagcttaaacagcacttttaattaactcctaaacattacatggtgggactgtggagactccgttaagacaggagcttgtcagaggtggacaacacgaagatttcctttgcattttcagtaacctttggagcacacttctcttatttctcctagagccccgtgcccctctgaagtgttaccacaatctagcttacctagcagccgttgattgttttttttccatctgagcaaacaggtaatttaagcatttatctcccctttcactttcaaaaagaatcatcattgattattcttgtttcacatctgaggatgaaggctaacggctgtgcttgccctggttactggatgggattctctgctgggcgggtggggaagcagctctacccttccctacccctccctgccccaactctgacttcgaataagccttggtcctatccagaatacactggacaccaaggccaaggacgttcacgttctgccctcgctgggtccagctgactctagcgtctgcacagccttgtgtgactcgtggtgacctaacctgagaaagagtgcaatcagatgttaaagtaactaaatagactgcggcacttttttgctttaaactagatcatcttagatttgtcgataccttcgaaatttgatggtttcatcccaaatgactgcactatatgtatgcatacggccacttttgattgctgcgccccttctgagtagtctttgacaatgtgttgtgttccccgatgtcgacttgatttccttttagtagcatctctctcttccatgtcttgatgttatgcaggaagtacaaaagtactttaaaattttgttatgaaataaaaaaaaaaaggatgggttttgtaaaaataataaaaaaaatatttttagcagaacaggacttacagggtcattgtccccacaatgtgccagtcggctctttgcactcgccttgtcctatatatccgtacggaggtgtgcaatcctgtgtcagtcgccttgtgacactgaagtggatgagttatagaggaggccctcgaggctgacccaatacggttactaagggagactacagggatctcacgacaaacattctgatacaatactcaacctcggtatatatatatatatatatatatatatacatatatatatatgtataaatataagaatatcccagcggcactttatactgttcactgtacaaaagcttacagttttccacaaggactttaataactagctggggaaaagattatgtaattacttggggctctgcaggaccttctctgtccagcgccccctttctgttgtgcgattagttgtagctgccatgctcagaattgccttttgagagctgaagcaaggtgcttactgtcacctgatgccatacacatggtcccaggcccacacccggggggcctctgttcatagcggcacatgcatttccccaccgcgtcttgtctgcagcttcttggccaatgtagtaatgcttttagtagagtaataggtagtatcagtttggattcttattgttatcacctatgtacaatggagaggggttctaagcacaaatctgctgctcatgtaacggtggtacataatatcaaatcaaaagttatctgtgactatatatagggatcacaaagtgtcacatgttagaatgctgaccttccacatggggttattgtgagtcatcagagcatatttattataacttattgttcatattcatttctaagttaatttaagtaatcatttattaagacagaattttgtataaactatttattgtgctctctgtggaactgaagtttgatttatttttgtactacacggcatgggtttgttgacactttaattttgctataaatgtgtggaatcacaagttgctgtgatacttcatttttaaattgtgaactttgtacaaactttgtcatgctgatgtgaacacatcttactctgaataaaaaggtgttgccacgtttgtagcacgaagga

SEQ ID NO: 6 - DNA, 7104 bp, Artificial Sequence
  Inducing factor
gtctgctgcctgctgatggaggctgcgccgcatccaccaggcagggctctgcacgcatgcccgtcttccccggcgtttagtggtgtcaacttattaatttccatcttcgacttcaggcctgttagtttcctgtctgaaagcattattacttttgtccatgtggcaaaataattagcatacagatgagaagtggccccgtgtctgcgcgccgatagagagaagatgtgcggttcgaagttgattggatgaatggagaacaaatggtgggcgttcaaaagcattagaagagttttgtagtcgcggagcaggctggagaattttctgggggtcccacagaggctgcagctgaggtctgggtgtgtgcaattctcagctgggctttgccctacccaggttgacgactgaagaatcacagagtgtggaaaagaacacaaggagaaattggtgagaacatctgcctagcatctccaagtcctgtggagggagccagcagtgctggtccaaagaggacccagagagtgaactcagagtgaccacatctgatagaagaggggaagatgtagtttctgagtccagtccagtgtttgtgtctctcacattgtcaacaaaagaaaggctccagctgtccccacagacatatggatattccaggagccacgtaaagatggagaaatggaggcacagagaaattaagtgacttggccacagtcacaagctggggaggaccagggaaagcctagagagagctggctctgggcctgcatcctgcccacggagtcaccctgcctccgtcctcaggagagaaggcttcctacaagatggacgtggactctgaggagaagcgccatcgcacacggtccaaaggggttcgagttcctgtggagccagccatacaagagctgttcagctgtcccactccaggctgcgacggcagtggtcacgtcagtggcaaatatgcacgacacagaagtgtatatggttgtcccttggctaaaaaaagaaaaacgcaagataaacagccccaagaacctgctcccaagcgaaaaccatttgcagtaaaagcagatagttcctcagtagacgaatgttatgagagtgatggtactgaagacatggatgataaggaggaagatgatgatgaggagttctctgaagacaatgatgagcaaggggatgatgacgacgaagatgaggtggatcgggaagacgaggaggagatcgaggaggaagatgatgaagaagatgatgatgatgaagatggtgacgatgtagaagaggaagaagaggatgatgatgaagaggaggaagaagaggaagaggaagaagaaaatgaagaccatcaaatgagttgtactcgaataatgcaggacacagacaaggatgataacaacaatgatgagtatgataactatgatgaactggtagctaagtcgctattaaatcttggcaaaattgctgaggatgcagcataccgagccaggactgaatcagagatgaacagcaatacctccaatagtctggaggacgatagtgacaaaaacgaaaacctcggtcggaaaagcgaactgagtctagacttagacagtgatgttgttagagaaacagtggactcccttaagctgttagcacaaggacatggtgttgtgctatcagagaatatcagtgacagaagttatgctgaggggatgtcacagcaggacagtagaaatatgaactatgtcatgctagggaagcccatgaacaatggactcatggagaagatggtggaggagagtgatgaggaagtgtgtctaagtagtctagagtgcctgaggaaccagtgctttgacctggccaggaaactcagcgagaccaacccacaggacaggagtcagccacccaacatgagtgtgcgccaacatgtccggcaagaggacgacttccctgggaggacgccagacaggagctactcggatatgatgaaccttatgcggctggaggagcagctcagtcccaggtctagaacgttctccagctgtgccaaggaggatgggtgtcatgagagggatgatgacaccacctcagtgaactcagacaggtctgaggaagtgtttgacatgaccaagggcaacctgactctgctagagaaagccattgccttggagacagagagagccaaggccatgcgggagaagatggccatggatgctgggagaagggataacctgagatcctatgaggaccagtctccaagacagctggctggggaagacagaaaatccaaatccagtgacagccatgtcaaaaagccatactatgatccctcaagaacagaaaagagagagagcaagtgtccaacccccgggtgtgatggaaccggccacgtaactgggctttacccgcatcaccgcagtctgtctggatgcccgcacaaagatagggtccctccagaaattcttgccatgcatgaaaatgttctcaagtgtcccactccaggctgcacagggcgagggcatgtgaatagcaacaggaactcgcacagaagcctctctggatgccccattgctgctgcagaaaaactggcaaaggcccaagagaaacaccagagctgtgatgtgtccaaatccaaccaggcctcagaccgagtcctcaggccaatgtgctttgtcaaacagcttgagattcctcagtatggctacagaaacaatgttcccacaaccacaccacgctccaacctggccaaggagcttgagaaatactccaagacttcgtttgagtacaacagttacgacaaccatacttatggcaaaagagccatagctcccaaggtgcaaaccagggacatatcccccaaaggatatgacgatgccaagcggtactgcaagaatgccagccccagcagcagcaccaccagcagctatgcacctagcagcagcagcaacctcagctgtggtggtggcagcagcgccagtagcacgtgtagcaagagcagctttgactacacacatgacatggaggccgcacacatggcagccacagccattctcaacctgtccacacgttgtcgtgaaatgccacagaacctgtccaccaagccacaggacctgtgtactgcccggaacccagacatggaggtggatgagaatggcaccctggacctgagcatgaacaagcagaggcctcgagacagctgctgcccagtcctgacacccctggaacccatgtctccgcagcagcaggccgtgatgagcagccgatgcttccagctgagcgagggggattgctgggacttgcctgtagactacaccaaaatgaagcctcggagggtagatgaggatgagcccaaagagattaccccagaagacttggacccattccaggaggctctggaagaaagacggtatccaggggaggtgaccatcccaagccccaaacccaagtaccctcagtgcaaggaaagcaaaaaggacttaataactctgtctggctgccccctggcggacaaaagcattcgaagtatgctggccaccagttcccaagagctcaagtgccccacccctggctgtgacggttctggacacatcactggcaattacgcttctcatcgaagcctttctgggtgcccgagagcaaagaagagtggcatccggatagcacagagcaaagaggacaaggaagaccaggagccaatcaggtgtccggtacctggctgtgacggtcagggacacatcactgggaagtatgcatcccaccgcagcgcctccgggtgtcccttggcagccaagaggcagaaagatgggtaccttaatggctcccagttctcctggaagtcggtcaagacggagggcatgtcctgccctacccccgggtgtgatgggtcaggacacgtcagtggcagcttcctcacacaccgcagcttgtcaggatgtccaagagccacatcagcaatgaagaaagcaaagctgtctggagaacagatgttgactatcaagcagcgagccagcaacggtatagaaaatgatgaagaaatcaagcagttagatgaagagatcaaggagcttaatgagtccaattcccagatggaggctgacatgatcaaactcagaactcagatcaccacaatggagagcaacctgaagacgattgaggaggagaacaaagtcattgaacagcagaatgagtcgctcttgcacgagttggccaacctgagccagtccctgatccacagcctcgccaacatccagctgcctcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctgaacagctgctctagtggtgactcatgcttaaaaaggatgcctcttgtttcttgctgctgtaacttaccagaaagtgttatatatttatttctgtcggaacagtgttatgctacaagacttcataatggttttgtgtgctctcgagagagtacctgcagactagttttggatacattcacattttgtacgttttcatataagctgacatagtgtgatttgccatgtaatgtttatagctgctgctgtctgcacatttgggggtctctatatttctgaagaggtaagctgatgaaaataaatagagtgtaaattctttttaatgctttagtgattaaatgttttagtattttgaactgaaatggacaaaaaaagaaaaaggaaaaaaaaagcaggtttgaacgatcactttgtggcctcctggccacttttagacattatcattttgcctcaggttggaggactttgtggaatttaagaaatacattttgtgtgcatattgtttcatagcaagaattggttgcaaaaaaatgctttatttttgaacaatgcttggaaatattatgtgacttttttgtttgtttgttttaggaggatggtgtatggtgggggcaataaatgaggttttttgcattccaaggaaatggcatatggattaactataagaaatgaaataagtaatttattgtaagacaacatcaagccatggaaacttggcagaagattcaaagcagcttaaacagcacttttaattaactcctaaacattacatggtgggactgtggagactccgttaagacaggagcttgtcagaggtggacaacacgaagatttcctttgcattttcagtaacctttggagcacacttctcttatttctcctagagccccgtgcccctctgaagtgttaccacaatctagcttacctagcagccgttgattgttttttttccatctgagcaaacaggtaatttaagcatttatctcccctttcactttcaaaaagaatcatcattgattattcttgtttcacatctgaggatgaaggctaacggctgtgcttgccctggttactggatgggattctctgctgggcgggtggggaagcagctctacccttccctacccctccctgccccaactctgacttcgaataagccttggtcctatccagaatacactggacaccaaggccaaggacgttcacgttctgccctcgctgggtccagctgactctagcgtctgcacagccttgtgtgactcgtggtgacctaacctgagaaagagtgcaatcagatgttaaagtaactaaatagactgcggcacttttttgctttaaactagatcatcttagatttgtcgataccttcgaaatttgatggtttcatcccaaatgactgcactatatgtatgcatacggccacttttgattgctgcgccccttctgagtagtctttgacaatgtgttgtgttccccgatgtcgacttgatttccttttagtagcatctctctcttccatgtcttgatgttatgcaggaagtacaaaagtactttaaaattttgttatgaaataaaaaaaaaaaggatgggttttgtaaaaataataaaaaaaatatttttagcagaacaggacttacagggtcattgtccccacaatgtgccagtcggctctttgcactcgccttgtcctatatatccgtacggaggtgtgcaatcctgtgtcagtcgccttgtgacactgaagtggatgagttatagaggaggccctcgaggctgacccaatacggttactaagggagactacagggatctcacgacaaacattctgatacaatactcaacctcggtatatatatatatatatatatatatatacatatatatatatgtataaatataagaatatcccagcggcactttatactgttcactgtacaaaagcttacagttttccacaaggactttaataactagctggggaaaagattatgtaattacttggggctctgcaggaccttctctgtccagcgccccctttctgttgtgcgattagttgtagctgccatgctcagaattgccttttgagagctgaagcaaggtgcttactgtcacctgatgccatacacatggtcccaggcccacacccggggggcctctgttcatagcggcacatgcatttccccaccgcgtcttgtctgcagcttcttggccaatgtagtaatgcttttagtagagtaataggtagtatcagtttggattcttattgttatcacctatgtacaatggagaggggttctaagcacaaatctgctgctcatgtaacggtggtacataatatcaaatcaaaagttatctgtgactatatatagggatcacaaagtgtcacatgttagaatgctgaccttccacatggggttattgtgagtcatcagagcatatttattataacttattgttcatattcatttctaagttaatttaagtaatcatttattaagacagaattttgtataaactatttattgtgctctctgtggaactgaagtttgatttatttttgtactacacggcatgggtttgttgacactttaattttgctataaatgtgtggaatcacaagttgctgtgatacttcatttttaaattgtgaactttgtacaaactttgtcatgctgatgtgaacacatcttactctgaataaaaaggtgttgccacgtttgtagcacgaagga

SEQ ID NO: 7 - DNA, 7189 bp, Artificial Sequence
  Inducing factor
gtggagctgcgtgcattaggggctggctgtggaggtaaagcagatctcttaggcccgctagcctctcctgctgcatgtgtcattcctgcccgcgcgacattttccccctaagctactgcacacaaaacagagcgagaaagcttctccctgcagtcttcttggaggcctcctggtttctcacccattgttggtgggtgtatttcaattttttgattccctggactgtgggttatgaaatcaatgctgctgaagacaaaagcaaccttccctgcctctcggtgctatgcgtggtcctcctcggcctcccacttgtgggggaaagggttttctctttctttctgtgtgttttgagccagcacagttaccaaaattgaacttgccgtcacttgtgagcggtgtggtcatggtgtgaggggtcccacagaggctgcagctgaggtctgggtgtgtgcaattctcagctgggctttgccctacccaggttgacgactgaagaatcacagagtgtggaaaagaacacaaggagaaattggtgagaacatctgcctagcatctccaagtcctgtggagggagccagcagtgctggtccaaagaggacccagagagtgaactcagagtgaccacatctgatagaagaggggaagatgtagtttctgagtccagtccagtgtttgtgtctctcacattgtcaacaaaagaaaggctccagctgtccccacagacatatggatattccaggagccacgtaaagatggagaaatggaggcacagagaaattaagtgacttggccacagtcacaagctggggaggaccagggaaagcctagagagagctggctctgggcctgcatcctgcccacggagtcaccctgcctccgtcctcaggagagaaggcttcctacaagatggacgtggactctgaggagaagcgccatcgcacacggtccaaaggggttcgagttcctgtggagccagccatacaagagctgttcagctgtcccactccaggctgcgacggcagtggtcacgtcagtggcaaatatgcacgacacagaagtgtatatggttgtcccttggctaaaaaaagaaaaacgcaagataaacagccccaagaacctgctcccaagcgaaaaccatttgcagtaaaagcagatagttcctcagtagacgaatgttatgagagtgatggtactgaagacatggatgataaggaggaagatgatgatgaggagttctctgaagacaatgatgagcaaggggatgatgacgacgaagatgaggtggatcgggaagacgaggaggagatcgaggaggaagatgatgaagaagatgatgatgatgaagatggtgacgatgtagaagaggaagaagaggatgatgatgaagaggaggaagaagaggaagaggaagaagaaaatgaccatcaaatgagttgtactcgaataatgcaggacacagacaaggatgataacaacaatgatgagtatgataactatgatgaactggtagctaagtcgctattaaatcttggcaaaattgctgaggatgcagcataccgagccaggactgaatcagagatgaacagcaatacctccaatagtctggaggacgatagtgacaaaaacgaaaacctcggtcggaaaagcgaactgagtctagacttagacagtgatgttgttagagaaacagtggactcccttaagctgttagcacaaggacatggtgttgtgctatcagagaatatcagtgacagaagttatgctgaggggatgtcacagcaggacagtagaaatatgaactatgtcatgctagggaagcccatgaacaatggactcatggagaagatggtggaggagagtgatgaggaagtgtgtctaagtagtctagagtgcctgaggaaccagtgctttgacctggccaggaaactcagcgagaccaacccacaggacaggagtcagccacccaacatgagtgtgcgccaacatgtccggcaagaggacgacttccctgggaggacgccagacaggagctactcggatatgatgaaccttatgcggctggaggagcagctcagtcccaggtctagaacgttctccagctgtgccaaggaggatgggtgtcatgagagggatgatgacaccacctcagtgaactcagacaggtctgaggaagtgtttgacatgaccaagggcaacctgactctgctagagaaagccattgccttggagacagagagagccaaggccatgcgggagaagatggccatggatgctgggagaagggataacctgagatcctatgaggaccagtctccaagacagctggctggggaagacagaaaatccaaatccagtgacagccatgtcaaaaagccatactatgatccctcaagaacagaaaagagagagagcaagtgtccaacccccgggtgtgatggaaccggccacgtaactgggctttacccgcatcaccgcagtctgtctggatgcccgcacaaagatagggtccctccagaaattcttgccatgcatgaaaatgttctcaagtgtcccactccaggctgcacagggcgagggcatgtgaatagcaacaggaactcgcacagaagcctctctggatgccccattgctgctgcagaaaaactggcaaaggcccaagagaaacaccagagctgtgatgtgtccaaatccaaccaggcctcagaccgagtcctcaggccaatgtgctttgtcaaacagcttgagattcctcagtatggctacagaaacaatgttcccacaaccacaccacgctccaacctggccaaggagcttgagaaatactccaagacttcgtttgagtacaacagttacgacaaccatacttatggcaaaagagccatagctcccaaggtgcaaaccagggacatatcccccaaaggatatgacgatgccaagcggtactgcaagaatgccagccccagcagcagcaccaccagcagctatgcacctagcagcagcagcaacctcagctgtggtggtggcagcagcgccagtagcacgtgtagcaagagcagctttgactacacacatgacatggaggccgcacacatggcagccacagccattctcaacctgtccacacgttgtcgtgaaatgccacagaacctgtccaccaagccacaggacctgtgtactgcccggaacccagacatggaggtggatgagaatggcaccctggacctgagcatgaacaagcagaggcctcgagacagctgctgcccagtcctgacacccctggaacccatgtctccgcagcagcaggccgtgatgagcagccgatgcttccagctgagcgagggggattgctgggacttgcctgtagactacaccaaaatgaagcctcggagggtagatgaggatgagcccaaagagattaccccagaagacttggacccattccaggaggctctggaagaaagacggtatccaggggaggtgaccatcccaagccccaaacccaagtaccctcagtgcaaggaaagcaaaaaggacttaataactctgtctggctgccccctggcggacaaaagcattcgaagtatgctggccaccagttcccaagagctcaagtgccccacccctggctgtgacggttctggacacatcactggcaattacgcttctcatcgaagcctttctgggtgcccgagagcaaagaagagtggcatccggatagcacagagcaaagaggacaaggaagaccaggagccaatcaggtgtccggtacctggctgtgacggtcagggacacatcactgggaagtatgcatcccaccgcagcgcctccgggtgtcccttggcagccaagaggcagaaagatgggtaccttaatggctcccagttctcctggaagtcggtcaagacggagggcatgtcctgccctacccccgggtgtgatgggtcaggacacgtcagtggcagcttcctcacacaccgcagcttgtcaggatgtccaagagccacatcagcaatgaagaaagcaaagctgtctggagaacagatgttgactatcaagcagcgagccagcaacggtatagaaaatgatgaagaaatcaagcagttagatgaagagatcaaggagcttaatgagtccaattcccagatggaggctgacatgatcaaactcagaactcagatcaccacaatggagagcaacctgaagacgattgaggaggagaacaaagtcattgaacagcagaatgagtcgctcttgcacgagttggccaacctgagccagtccctgatccacagcctcgccaacatccagctgcctcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctgaacagctgctctagtggtgactcatgcttaaaaaggatgcctcttgtttcttgctgctgtaacttaccagaaagtgttatatatttatttctgtcggaacagtgttatgctacaagacttcataatggttttgtgtgctctcgagagagtacctgcagactagttttggatacattcacattttgtacgttttcatataagctgacatagtgtgatttgccatgtaatgtttatagctgctgctgtctgcacatttgggggtctctatatttctgaagaggtaagctgatgaaaataaatagagtgtaaattctttttaatgctttagtgattaaatgttttagtattttgaactgaaatggacaaaaaaagaaaaaggaaaaaaaaagcaggtttgaacgatcactttgtggcctcctggccacttttagacattatcattttgcctcaggttggaggactttgtggaatttaagaaatacattttgtgtgcatattgtttcatagcaagaattggttgcaaaaaaatgctttatttttgaacaatgcttggaaatattatgtgacttttttgtttgtttgttttaggaggatggtgtatggtgggggcaataaatgaggttttttgcattccaaggaaatggcatatggattaactataagaaatgaaataagtaatttattgtaagacaacatcaagccatggaaacttggcagaagattcaaagcagcttaaacagcacttttaattaactcctaaacattacatggtgggactgtggagactccgttaagacaggagcttgtcagaggtggacaacacgaagatttcctttgcattttcagtaacctttggagcacacttctcttatttctcctagagccccgtgcccctctgaagtgttaccacaatctagcttacctagcagccgttgattgttttttttccatctgagcaaacaggtaatttaagcatttatctcccctttcactttcaaaaagaatcatcattgattattcttgtttcacatctgaggatgaaggctaacggctgtgcttgccctggttactggatgggattctctgctgggcgggtggggaagcagctctacccttccctacccctccctgccccaactctgacttcgaataagccttggtcctatccagaatacactggacaccaaggccaaggacgttcacgttctgccctcgctgggtccagctgactctagcgtctgcacagccttgtgtgactcgtggtgacctaacctgagaaagagtgcaatcagatgttaaagtaactaaatagactgcggcacttttttgctttaaactagatcatcttagatttgtcgataccttcgaaatttgatggtttcatcccaaatgactgcactatatgtatgcatacggccacttttgattgctgcgccccttctgagtagtctttgacaatgtgttgtgttccccgatgtcgacttgatttccttttagtagcatctctctcttccatgtcttgatgttatgcaggaagtacaaaagtactttaaaattttgttatgaaataaaaaaaaaaaggatgggttttgtaaaaataataaaaaaaatatttttagcagaacaggacttacagggtcattgtccccacaatgtgccagtcggctctttgcactcgccttgtcctatatatccgtacggaggtgtgcaatcctgtgtcagtcgccttgtgacactgaagtggatgagttatagaggaggccctcgaggctgacccaatacggttactaagggagactacagggatctcacgacaaacattctgatacaatactcaacctcggtatatatatatatatatatatatatatacatatatatatatgtataaatataagaatatcccagcggcactttatactgttcactgtacaaaagcttacagttttccacaaggactttaataactagctggggaaaagattatgtaattacttggggctctgcaggaccttctctgtccagcgccccctttctgttgtgcgattagttgtagctgccatgctcagaattgccttttgagagctgaagcaaggtgcttactgtcacctgatgccatacacatggtcccaggcccacacccggggggcctctgttcatagcggcacatgcatttccccaccgcgtcttgtctgcagcttcttggccaatgtagtaatgcttttagtagagtaataggtagtatcagtttggattcttattgttatcacctatgtacaatggagaggggttctaagcacaaatctgctgctcatgtaacggtggtacataatatcaaatcaaaagttatctgtgactatatatagggatcacaaagtgtcacatgttagaatgctgaccttccacatggggttattgtgagtcatcagagcatatttattataacttattgttcatattcatttctaagttaatttaagtaatcatttattaagacagaattttgtataaactatttattgtgctctctgtggaactgaagtttgatttatttttgtactacacggcatgggtttgttgacactttaattttgctataaatgtgtggaatcacaagttgctgtgatacttcatttttaaattgtgaactttgtacaaactttgtcatgctgatgtgaacacatcttactctgaataaaaaggtgttgccacgtttgtagcacgaagga

SEQ ID NO: 8 - DNA, 7152 bp, Artificial Sequence
  Inducing factor
taagctactgcacactaaacagtgagagagcttttccctgcagtcttgttgaagcaccccgggttttttgctcattgttggtgggtgcattttaattttttcattccctggactatgggttatgatatccatactcactgaagacaaaaagccaccttttctgcgtcttggtggcatgcatgtgtctcatcatcctttcaaacttgtggtggaacagggttttcttccctgtctgtgtattttgagccagcacagttaccaaaattgaacttgtctttcgcttgtgagcggttgtggtcattgtgagggcgggtcatgaggaggctgtagccaaggacgaggtgtgtgcggctgttgcctggacgtttgtccaatccacgttgacatttgagggatcacagcgtgtgaaaatgaactcagaggagaattggtgaattcctatccagtgggcatcttcaaaccctggtcgacggcggaagaatatcaggtcctgagatcacccacccggcgcggcaacagtgcagagtggccacatctggtggaagaagaaaaaaatgtagttattgaattcaatcaagtgtttgcatctttcaagctatcaacaaaattccatcaagaaaggttccagttggtctcacagacgtatggatatccgaggagccacctaaagatggagaaatcaaggcatagagagattaagtgactttgccacagtcacaagctggagaggaccaggagtagagcttagagcgagcccctgactctgggcctgcgtcctgccaggagtcacgctgcctccgttcctaggagagaagacttcctgtaagatggaggtggacaccgaggagaagcggcatcgcacgcggtccaaaggggttcgagttcccgtggaaccagccatacaagagctgttcagctgtcccacccctggctgtgacggcagtggtcatgtcagtggcaaatatgcaagacacagaagtgtatatggttgtcccttggcgaaaaaaagaaaaacacaagataaacagccccaggaacctgctcctaaacgaaagccatttgccgtgaaagcagacagctcctcagtggatgagtgtgacgacagtgatgggactgaggacatggatgagaaggaggaggatgagggggaggagtactccgaggacaatgacgagccaggggatgaggacgaggaggacgaggagggggaccgggaggaggaggaggagatcgaggaggaggatgaggacgatgacgaggatggagaagatgtggaggatgaagaagaggaagaggaggaggaggaggaggaggaagaggaagaagaaaacgaagaccatcaaatgaattgtcacaatactcgaataatgcaagacacagaaaaggatgataacaataatgacgaatatgacaattacgatgaactggtggccaagtcattgttaaacctcggcaaaatcgctgaggatgcagcctaccgggccaggactgagtcagaaatgaacagcaatacctccaatagtctggaagacgatagtgacaaaaacgaaaacctgggtcggaaaagtgagttgagtttagacttagacagtgatgttgttagagaaacagtggactcccttaaactattagcccaaggacacggtgttgtgctctcagaaaacatgaatgacagaaattatgcagacagcatgtcgcagcaagacagtagaaatatgaattacgtcatgttggggaagcccatgaacaacggactcatggaaaagatggtggaggagagcgatgaggaggtgtgtctgagcagtctggagtgtttgaggaatcagtgcttcgacctggccaggaagctcagtgagaccaacccgcaggagaggaatccgcagcagaacatgaacatccgtcagcatgtccggccagaagaggacttccccggaaggacgccggacagaaactactcggacatgctgaacctcatgcggctggaggagcagttgagcccccggtcgagagtgtttgccagctgtgcgaaggaggatgggtgtcatgagcgggacgacgataccacctctgtgaactcggacaggtctgaagaggtgttcgacatgaccaaggggaacctgaccctgctggagaaagccatcgctttggaaacggaaagagcaaaggccatgagggagaagatggccatggaagctgggaggagggacaatatgaggtcatatgaggaccagtctccgagacaacttcccggggaggacagaaagcctaaatccagtgacagccatgtcaaaaagccatactatgatccctcaagaacagaaaagaaagagagcaagtgtccaacccccgggtgtgatggaaccggccacgtaactgggctgtacccacatcaccgcagcctgtccggatgcccgcacaaagatagggtccctccagaaatccttgccatgcatgaaagtgtcctcaagtgccccactccgggctgcacggggcgcgggcatgtcaacagcaacaggaactcccaccgaagcctctccggatgcccgatcgctgcagcagagaaactggccaaggcacaggaaaagcaccagagctgcgacgtgtccaagtccagccaggcctcggaccgcgtgctcaggccaatgtgctttgtgaagcagctggagattcctcagtatggctacagaaacaatgtccccacaactacgccgcgttccaacctggccaaggagctcgagaaatattccaagacctcgtttgaatacaacagttacgacaaccatacttatggcaagcgagccatagctcccaaggtgcaaaccagggatatatcccccaaaggatatgatgatgcgaagcggtactgcaaggaccccagccccagcagcagcagcaccagcagctacgcgcccagcagcagcagcaacctgagctgcggcgggggcagcagcgccagcagcacgtgcagcaagagcagcttcgactacacgcacgacatggaggcggcccacatggcggccaccgccatcctcaacctgtccacgcgctgccgcgagatgccgcagaacctgagcaccaagccgcaggacctgtgcgccacgcggaaccctgacatggaggtggatgagaacgggaccctggacctcagcatgaacaagcagaggccgcgggacagctgctgccccatcctgacccctctggagcccatgtccccccagcagcaggcagtgatgaacaaccggtgtttccagctgggcgagggcgactgctgggacttgcccgtagactacaccaaaatgaaaccccggaggatagacgaggacgagtccaaagacattactccagaagacttggacccattccaggaggctctagaagaaagacggtatcccggggaggtgaccatcccaagtcccaaacccaagtaccctcagtgcaaggagagcaaaaaggacttaataactctgtctggctgccccctggcggacaaaagcattcgaagtatgctggccaccagctcccaagaactcaagtgccccacgcctggctgtgatggttctggacatatcaccggcaattatgcttctcatcggagcctttcaggttgcccaagagcaaagaaaagtggtatcaggatagcacagagcaaagaagataaagaagatcaagaacccatcaggtgtccggtccccgggtgcgacggccagggccacatcactgggaagtacgcgtcccatcgcagcgcctccgggtgccccttggcggccaagaggcagaaagacgggtacctgaatggctcccagttctcctggaagtcggtcaagacggaaggcatgtcctgccccacgccaggatgcgacggctcaggccacgtcagcggcagcttcctcacacaccgcagcttgtcaggatgcccgagagccacgtcagcgatgaagaaggcaaagctttctggagagcagatgctgaccatcaaacagcgggccagcaacggtatagaaaatgatgaagaaatcaaacagttagatgaagaaatcaaggagctaaatgaatccaattcccagatggaagccgatatgattaaactcagaactcagattaccacgatggagagcaacctgaagaccatcgaagaggagaacaaagtgattgagcagcagaacgagtctctcctccacgagctggcgaacctgagccagtctctgatccacagcctggctaacatccagctgccgcacatggatccaatcaatgaacaaaattttgatgcttacgtgactactttgacggaaatgtatacaaatcaagatcgttatcagagtccagaaaataaagccctactggaaaatataaagcaggctgtgagaggaattcaggtctgaacagctgctgtagtgatgaaactcttgcttaaaaaggatgcctcttgttttttgctgctgtaacttaccagaaagtgttctatatttatttctgtttgaatttgaaacagtgttatgcttacaagacttcataatgattttatgtcttgctttaaagatagtacctgcagaatagtttttgaatacacccacattttgtacgtttccatgtaagctgacatagtgttctgccatgtaatgtttatagctgctgatgtatgcacatttgggggtatatctatttctgaagaggtaagctgatcaaaataaatagagtgtaaattctttttaatgctttagtgattaaatgttttagtattttgaactgaaatggacacacaaacacacacacgcacacacagacccacagctttgaatgatcatgttgtggctgagcagccgctttttagacgttatcattttgcctcatgttggaggactttatggaatttaagaaatacattttgtgtgcatattgtttcatagcaagaattcgttgcaaaaatgctttatttttgaacaatgcttggaaatattatgtgacttttttgtttgtttgttttaggaggatggtgtatggtgggggcaataaatgaggttttttgcattccaaggaaatggcatatggattaactgtaagaaatgaaataagtaatttattgtaagacaacatcaagccatggaaacttggcagaagattcaaagcagcttaaacagcacttttaaattaactcctaagcgttacatggttgtgactatggaaactccagttaagacaggatcttatcagaggtggacaacgtgaagatttccttttccattttcaataaactttggaacaaccttctcgtatctcccctagagtttcgtgcccctctgaactgtctgttattgcaatgtagtttatcaacagaatttgtgtgttttcgatttaagctaaaagataatttaagaacatttatttccccttttcactttaaaaaattatgattattcctattattgttatgaaccttcttattttacatttgagggataaaggcaaatgatttgtgagtcttctagttactggaccgagttttctgctggatctggtgggaaggcagctcggtaaagtttccctcctgctccccccgcccgactttgactctgaatcagcatttggtcctattcagaggactcttaccacgacgtttctgttctacacttgggtggagaccagttgaccatagagcatttgcagagcctcattgtttgatttcttgtgactattctaagaatgaatgcaatcagattttaaaagtaactaaatatacttcagcacttttttgctttaaactagatcatcttagacttgtttataccttccagatttgattgttttactcccaatgactgcactatatgtatgcataagaccacttttgagcgctgtgttcccccttctgagtagtcctttgacgacgtgttgtgttttctgatgttgacttgagttccatttagtagcatctcttccttccatgtcttgatgttatgcaggaagtacagacgtactttaaatttttgttatgaaataaaaaaaagatgggttttgtaaaaataaaaaaaaaatatttttagcagaacaggacttacagggtcattgtccccacaatgtgccagtcgactatttgcacttaccttgtcctatatatccgtacggaggtgtgcaattcctcgtgtcagtagccttgtgacactgaacctggatggattatagaggagccctcacggctgatcaataatgttgcaaagggagactacagggatctcacgacgaatattctgatacaatactcaacctcggtatatatatatgtgtataaatatatgtatatcccagcggcactttatactgttcactgtacaaaagcttacagttttccacaaggactttaataactagctgggaaaagacgatgtaattatttcggggctctgcggaaccttctctgtacagcgccccctttctgttgtgctattggttgcagctgccatgctcagaatgcgttttgagagctgaagcaaggtgcttgcagtcacctgaggccgtccgtgtggcccagggccccagctgcctttagggcccccattgttcataacagcatatgcatttccccaccgcgttgtgtctgcagcttctttgccaatatagtaatgcttttagtagagtactagatagtatcagttttggattcttattgttatcacctatgtacaatggaaagggattttaagcacaaacctgctgctcatctaacgttggtacataatctcaaatcaaaagttatctgtgactattatatagggatcacaaaagtgtcacatattagaatgctgacctttcatatggattattgtgagtcatcagagtttattataacttattgttcatattcatttctaagttaatttaagtaatcatttattaagacagaattttgtataaactatttattgtgctctctgtggaactgaagtttgatttatttttgtactacacggcatgggtttgttgacactttaattttgctataaatgtgtggaatcacaagttgctgtgatacttcatttttaaattgtgaactttgtacaaattttgtcatgctggatgttaacacatcttactctaaataaacaaggtgttgccacatttgtagcacgaaggatctctaaaaaaaaaaaaaaaaaa

SEQ ID NO: 9 - DNA, 2492 bp, Artificial Sequence
  Inducing factor
ggcagacaatcgtaaagctgctcgggggtcgctcacctgctcccgcctcctggagcccctaccgggtctgaccgctcttgccccctccattgcccgccccccgcccgagccgggccaggagccgagaggtccgagaggcccgggaaggatgcggccagcagctgcggtcccgagccacgcacggtgatcgcctcctccaggggaggaggcgcctagaccgtagcctcatttcttgttttcctctccctccccctccccgtaattacattggttgctggaagggacccggagagatagacagaggaggcgcctcgcatccccctctcgcccgccacccgtgctctttcctgtcccgggtcaggatgactggagtctttgacagtctggtggctgatatgcactcgacccagatcaccgcctccagcacgtaccaccagcaccagcagcccccgagcggtgcgggcgccggccctggcggcaacagcaacagcagcagcagcaacagcagcctgcacaagccccaggagtcgccaaccctcccggtgtccacggctacggacagcagctactacaccaaccagcagcacccggcgggcggcggcggcgggggggcctcgccctacgcgcacatgggctcctaccagtaccacgccagcggcctcaacaatgtctcctactccgccaaaagcagctacgacctgggctacaccgccgcgtacacctcctacgcgccctacggcaccagttcgtctccggtcaacaacgagccggacaaggaagaccttgagcctgaaatccgaatagtgaacgggaagccaaagaaagtccggaaaccacgcaccatctactccagtttccagctggcggcccttcaacgacgcttccagaagacccagtatctggccctgccagagcgagccgagctggcggcgtccctgggcctcacccaaactcaggtcaaaatctggttccagaaccgccgatccaagttcaagaagatgtggaaaagcggcgagatacccaccgagcagcaccctggagccagcgcttctcctccttgtgcctccccgccggtctcggcgccagcatcctgggacttcggcgcgccgcagcggatggctggcggcggcccgggcagcggaggcggcggtgcgggcagctctggctccagcccgagcagcgccgcctcggcctttctgggaaactacccgtggtaccaccaggcttcgggctccgcttcacacctgcaggccacagcgccacttctgcatccttcgcagactccgcaggcgcaccatcaccaccatcaccaccaccacgcaggcgggggcgccccggtgagcgcggggacgattttctaacctcgagtggacagcgtctgagacttgagaactgagacaccgtgttcttcagcacgcgtcctgtagctagaggctcgaacctaccagcccgcgaccgccacccacccagcggtattggaagtggcgaccaggggtgacccgtcgtggagccacttctcgcactgccacggagcagccggaaacaaggaggcctcgtgggatcctttagtgccaagcctggcgtccctggcctcaattccgacctgagccccggccacttttaggccatcctcggacttgtcgccccttcccagccccctcagcgtgatcctcagggtccttggtctcttcaggccctgagcggggtcaggcactgggacgccttccccgccctcgaggcttcccggctctcagaggctccttttttctctcctgctccaggcctcagctccagtccgcttcggttattgacctcctgggtcctactccaccctatcacctacccccgtgacccccgggatcccgccggttttttcccctcctgggggctcatgggggagaattcccagctctcccaactcttcccgcctcccactttccccaggaacactcccgtagctccttcatccattgccagtggagggtttttatttttatttttttaaaagtttaggtgcctttgcggatgacctcattttgatgttaaaaaaaaactgatttttttaatatgtggacgctgcaaaaaatgtgtttaaattatcttttttaagatgaattaaggactattagctcgaacttcgatgcagagtttgtaaataagggtgtctgtgcagatttccccacatttatttgtataaaaatttctcatcttttcagacttttttgtaaaccgcccccgagttgtgaaagctgcgacgtagcagtgacctcagcagtccctatgttttccccctttgaaaatttttctcagttaaattaagctactgatttgaatttgtttttatcttgtcctaaagtctttgttcttgaaatgcaaggtatttgggggttatttattatgaaagcaacatactcttaatgttgattttacaatatgaagagattatttaaaataaattattgttttcattggaaaaaaaaaaaaaaaaaaaa

SEQ ID NO: 10 - DNA, 2308 bp, Artificial Sequence
  Inducing factor
aagaggatgcgaccagaggcagctgtcccgagcagcgcacggtgatcgcctcccccggaggaggaggcgcctagacggttgcctcctttcttgtttccccaccaccacccccgtaattacattggctgctggaggggaccgggagagacagaggaggcgcctggcttcccccgcacgcccgccacctctgctctttcccgtctcgggccaggatgactggagtctttgacagtctagtggctgatatgcactcgacccagatcgccgcctccagcacgtaccaccagcaccagcagcccccgagcggcggcggcgccggcccgggtggcaacagcagcagcagcagcagcctccacaagccccaggagtcgcccacccttccggtgtccaccgccaccgacagcagctactacaccaaccagcagcacccggcgggcggcggcggcggcgggggctcgccctacgcgcacatgggttcctaccagtaccaagccagcggcctcaacaacgtcccttactccgccaagagcagctatgacctgggctacaccgccgcctacacctcctacgctccctatggaaccagttcgtccccagccaacaacgagcctgagaaggaggaccttgagcctgaaattcggatagtgaacgggaagccaaagaaagtccggaaaccccgcaccatctactccagtttccagctggcggctcttcagcggcgtttccaaaagactcaatacttggccttgccggagcgagccgagctggcggcctctctgggcctcacccagactcaggtcaaaatctggttccagaaccgccggtccaagttcaagaagatgtggaaaagtggtgagatcccctcggagcagcaccctggggccagcgcttctccaccttgtgcttcgccgccagtctcagcgccggcctcctgggactttggtgtgccgcagcggatggcgggcggcggtggtccgggcagtggcggcagcggcgccggcagctcgggctccagcccgagcagcgcggcctcggcttttctgggcaactacccctggtaccaccagacctcgggatccgcctcacacctgcaggccacggcgccgctgctgcaccccactcagaccccgcagccgcatcaccaccaccaccatcacggcggcgggggcgccccggtgagcgcggggacgattttctaaccccagggagaactcgccagagactgagagcagagaccacttatcctcattgcttaccccgagccggggttccctcctcccggcccgctgccgccacccacctctcctgcaggctgcgacctgcagtggcccgtctcaggccctgctcactcccggggccaccaaacgggcccctctctcgggggaaccggacagcagcttggcaaaggcctccctaaaaggccgcatttctgacctgagccccgggtctcggctgtttcgagccccgcctcggacttgccttccctcccctccgggtgagcctgtctggcgccttcctcgccccgggctgagagctgggtcccgggagatggaagcctcccaggcgcgcgaggcttcccgggcgctctgaggcttctttctcctcgcccgctcccctgggctcagctcggacgctgcagttattgacctcccggtcccgcctgcccgccctcccccacgtggccccttgacccgggcggccccgccgcttctttccttcctgcagttcccagccctcggagcccccatcccttatcttacccccaccgcgctcccccaggagcgctccctcagctctctcctcatccatcaccagtggagtttttttatttgttatttttttaaaagtttaggtgcctttgcggatgacctcattttgacgttgaaaaaatgattttttaatatgtgaacactgcaaaaatgtgtttaaattatcttttttaaaacctattcaggattattagcctggacttggacacagagtttgtaaataaaggtgtctgtgcagattttcccactgatttatttgtataaaaatactcatcttttcagacttttttgtaaacccccagttgtgaaaactgcagtttagcagtgacctcagcaacccctcctttttattttttcctttaaaaacatttcagttaaattaagctactgatttggatttgttttatcgtatcctaaagtctttgttgttgaaatgaaaggtattttggggttatttattatgaaaacaacatgctcttaatgttgattttacaatatgaagagattatttaaataaattattgttttcattggaaaaa